(19) United States
(12) Patent Application Publication
(10) Pub. No.: US 2025/0144203 A1
(43) Pub. Date: May 8, 2025

(54) NANOSTRUCTURE-BASED VECTOR SYSTEM FOR BACTERIAL AND VIRAL ANTIGENS

(71) Applicant: BIOSYNTH S.R.L., Rapolano Terme (SI) (IT)

(72) Inventor: Massimo PORRO, Rapolano Terme (SI) (IT)

(21) Appl. No.: 18/835,724

(22) PCT Filed: Feb. 2, 2023
(86) PCT No.: PCT/IB2023/050902

§ 371 Date: Aug. 3, 2024

Related U.S. Application Data
(60) Provisional application No. 63/306,459, filed on Feb. 3, 2022.

(51) Int. Cl. A61K 39/385; A61K 39/00; A61K 39/05; A61K 39/215; A61P 31/04

(52) U.S. Cl. CPC A61K 39/385; A61K 39/05; A61K 39/215; A61P 31/04; A61K 2039/6037; A61K 2039/64; C12N 2770/20034

(57) ABSTRACT

The present invention relates to novel nanostructure-based vector system mimicking the shape and the size-characteristics of a viral nanoparticle and presenting functional antigens expressed by different viruses or bacteria to the host's immune system through the presentation together with a helper T-cell dependent carrier protein against which the host's immune system is universally primed through the serological presence of carrier-specific antibodies for triggering an immediate boost of the human immune system. The invention further relates to vaccines based on such nanostructure-based vector system for the prevention of infection mediated by viral and bacterial pathogens in a mammal host.

### BACKGROUND ART

In the field of vaccines, one of the most important strategies for defining their composition and formulation is the way the selected antigens are presented to the host immune system.

Stability of the antigens, immunogenic potency of the antigens and immunological memory conferred by such antigens to the host are the key parameters concurring in defining the optimal composition and formulation of a given vaccine. The definition of all such parameters are dependent from the physical chemical characteristics of the antigens and therefore there is not a univocal strategy that may fit well for antigens of different nature, that is protein antigens or carbohydrate antigens or glycopeptide antigens as a few examples.

A modern strategy to efficiently convey antigens to the mammalian immune system is the one of using liposomes entrapping antigens that then become efficiently presented to the immune system. These micelle-based systems are often represented by nanostructures with size dimensions in the order of 20-200 nm and have been crucially helpful in the recent COVID-19 pandemic for properly conveying to the human immune system a variety of efficacious subunit protein-based and mRNA-based vaccines.

However, there are immunological properties that such modern technologies to vehicle vaccines cannot be yet considered definitively accomplished, that is their incapability to circumvent the early lag-phase of the immune system for immediately responding to the given antigen, because of the lacking antigenic imprinting of the novel antigen administered in the form of vaccine, for which an immunological memory does not exist in the host's immune system. For this reason, at least two/three vaccine doses are needed before an appropriate immunological, T-cell dependent, memory becomes established.

There is also a strong need of an immediate immunogenicity of vaccines in medical situations where vaccinal intervention is crucial as it is needed in emergency conditions.

This significant lack of immediate immunogenicity of vaccines has been responsible in the COVID-19 pandemic for delaying the level of protective antibodies in the population exposed to viral attacks over time while the herd immunity in the population also became largely postponed despite the mass immunization campaigns implemented worldwide.

During the COVID-19 pandemic, the immunological latency phase for reaching a sustained IgG antibody-mediated specific immune response in people, took at least two injections of any of the few kinds of vaccines available in a time interval of 4 to 6 weeks. Also, even in normal conditions of public health it is of practical and social value to reach the highest efficiency for a vaccination protocol as well seen in the nowadays post-pandemic situations where only a fraction of the eligible population receives one single dose of vaccine while the majority of it very often do not even receive a booster (e.g.: public data recorded in the USA for the fourth quarter of the 2022 related to the COVID-19 immunization show that 68% of the total population has been vaccinated with a single dose but only half of this percentage received a single boost). When in urgent situations as in pandemic outbreaks or in post-pandemic situations where the herd immunity must be assessed as quickly as possible, the possibility to immediately provide a boost to the host's immune system against the invading pathogen is of paramount importance to control the spreading of infection.

These experimental observations have induced the Applicant to rethink the way a given anti-infective vaccine can be properly formulated, in the most appropriate composition, in order to overcome the limits today existing in the immunogenic activity of novel vaccines as imposed by the lack of antigen-specific immunological memory in the host.

According to the above, the Applicant turned to consider the micelle-based system known as the Lipid A-SAEP (Synthetic Anti Endotoxin Peptide) complex (U.S. Pat. No. 5,652,211 in the name of BiosYnth) named Endotoxoid A, as a highly stable micellar system with dimensions in the range 10-100 nm which is here found to allow the incorporation of various bacterial and viral antigens on the basis of interacting strong hydrophobic forces. Such dimensions fall within the definition of nanoparticle according to the IUPAC terminology (2012) for biologically related polymers. Also, the size dimensions and the molecular characteristics of Endotoxoid A are resembling the ones of most patogenic viruses, and this observation has suggested to the Applicant that the mammalian immune system could have more possibilities for targeting it as foreign antigen for vaccinal use, with the parallel possibility of working as vector for antigens being inglobated into such Endotoxoid A structure. The further search for the enhancement of the immunogenic properties of a given vaccine has been then linked to certain characteristics of the antigens embedded into the Endotoxoid A nanostructure within which at least one of such antigens may work as helper T-cell dependent entity for immediately triggering a boosting activity on the host's immune system when such an antigen is antigenically cross-reactive with one or more of the antigens present in the paediatric vaccines universally used for establishing the antigen-specific imprinting in the early life of an individual which then lasts for the life-long existence of such human subject.

From the above considerations, the Applicant found that the Endotoxoid A could become a general system for vectoring any kind of antigens in a way that it may trigger an immediate (or booster) immune response in any individual which is “antigenically primed” against one of the antigen components of such Endotoxoid-based vector system, thus by-passing the initial lag-phase of the immune system which needs an “antigenic education” before mounting an efficient and specific immune response to novel antigens of viral and bacterial nature.

In the overall the invention relates to a novel concept developed for the design of subunit vaccines focused on conformational epitopes of functional antigens expressed by different viruses and bacteria carried by a helper T-cell dependent carrier protein against which the host's immune is universally primed through the serological presence of anti-carrier specific antibodies (i.e. using the carrier protein CRM197 in the composition of the VS when the host's immune system is primed towards diphtheria toxin/toxoid because of the DPT vaccination in the paediatric age) for triggering an immediate boost of the human immune system. The goal is achieved by conferring to these hybrid pluripotent antigens the property to acquire the helper T-cell dependent characteristics of the carrier protein, purposely presented to the human or animal host's immune system via the nanostructured vector Lipid A-SAEP complex (i.e. Endotoxoid A) for mimicking the shape and the size-characteristics of a viral nanoparticle.

Therefore, it is an object of the present invention a nanostructured vector system comprising a complex Lipid A-SAEP or a Lipid A derivative-SAEP derivative (Endotoxoid A) that incorporates at least one helper T-cell dependent carrier antigen. Said T-cell dependent carrier antigen having the antigenic features above reported relatively to the universal priming by DPT.

According to a preferred embodiment of the invention said helper T-cell dependent carrier antigen is a helper T-cell dependent carrier protein selected from the group consisting of CRM197, Diphtheria Toxoid, Tetanus Toxoid, Pertussis Toxoids or a combination thereof. Alternatively, their related rDNA protein derivatives may be used.

Alternatively, their related protein antigens obtained by the use of rDNA technology in the replacement of the well known classic process of 1) fermenting natural bacterial strains; 2)-purifying the secreted bacterial toxins and, finally; 3)-detoxifying such bacterial toxins by chemically denaturating methods (e.g.: formaldehyde treatment). For instance, the protein CRM197 (cross-reacting material 197) obtained by rDNA technology in a variety of bacterial cells is the antigenic cross-reactive but non-toxic version of the diphtheria toxin secreted by wild species of C. diphtheriae.

According to another alternative embodiment of the nanostructured vector system of the invention the Lipid A derivative is selected from the group of R-LPS, R/S-LPS or S-LPS chemotype and the SAEP derivative is a cationic and amphipathic peptide structure in a linear or cyclic conformation with a ratio between cationic and hydrophobic amino acids of ≥0.5.

According to another preferred embodiment of the nanostructured vector system of the invention the Lipid A derivative-SAEP derivative is Endotoxoid A.

In a preferred embodiment of the invention, the helper T-cell dependent carrier protein is CRM197, as the human immune system is universally primed against diphtheria toxin/toxoid through the serological presence of anti-diphtheria antibodies induced by the paediatric DPT vaccination in the early stage of life.

CRM197 is a helper-T cell dependent protein immunogenically cross-reactive with Diphtheria Toxin and Toxoid, which may be incorporated together with bacterial and/or viral antigens into the nanostructured vector system thus providing an efficient strategy for presenting the carried antigens to the immune system of mammalians and triggering an immediate cellular and humoral immune response.

This immunological property is achieved by making use of the carrier protein CRM197, a well-established helper T-cell dependent carrier of human glycoconjugate vaccines, which is known to be very efficient in all ranges of ages and especially in human infants (2-6 months old) where the immune system is still immature and in the senior population (>65 y old) where the immune system is weakening because of the age.

The principle of vectoring a variety of antigens herewith which is disclosed for providing an immediate immunological “booster” effect, lays in the fact that the human population is almost universally primed against Diphtheria Toxoid because of the paediatric vaccination with DT and DTP (Diphtheria Tetanus Pertussis) vaccines. Therefore, the protein CRM197 as cross-reactive antigen with Diphtheria Toxin/Toxoid can work as a perfect helper T-cell dependent carrier protein for providing a strong “booster” effect against the carried antigen/hapten just after the first dose of such an antigen is given to a mammalian host when administered in the form of an appropriate molecular composition involving its incorporation into the disclosed formulation of the nanostructured Vector System (VS).

According to a further embodiment the viral antigens belongs to one of the subunit (i.e. S1 or S2 subunit) of the spike glycoprotein of SARS-Cov-2 virus. In a preferred embodiment the viral antigen is the Receptor Binding Domain of the S1 subunit in the spike glycoprotein of SARS-CoV-2.

The nanostructured vector system according to the invention may further incorporate one or more bacterial and/or viral antigens, in conjugated or unconjugated form.

The bacterial and viral antigens incorporated into the micelles of the Endotoxoid A system may be natural molecular entities (e.g.: purified proteins, such as those derived from Diphtheria or Tetanus or Pertussis organisms, the natural glycoproteins and the derived complex carbohydrates) as well as synthetic and semi-synthetic molecular entities (e.g.: conjugated glycoprotein, synthetic peptides, synthetic lipopeptides, synthetic liposaccharides).

In a preferred embodiment of the invention, the bacterial antigens may comprise capsular polysaccharides and/or lipopolysaccharides, in conjugated or unconjugated form.

According to a preferred embodiment of the invention the viral antigens are viral conformationally-dependent antigens. These may be selected from the group comprising the envelope Glycoprotein E homodimer; the RSV perfusion F glycoprotein, or its antigenic conformational site phi containing the RSF trimer; the outer domain epitope of the Glycoprotein gp120; HA, NA, NP and M1 antigens; rabies glycoprotein trimer RABV-G; the glycoprotein trimer EBOV.

The nanostructured vector system may incorporate the viral antigens in conjugated or unconjugated form.

The amounts of bacterial and/or viral antigens to be incorporated are those effective for inducing an immune response when administered to a mammalian host including a human host or a veterinary.

In a preferred embodiment of the present invention the bacterial and/or viral antigens are conjugated to the helper T-cell dependent carrier antigen or protein. Preferably, the helper T-cell dependent carrier protein is CRM197.

The goal is achieved by conferring to these hybrid pluripotent antigens the property to acquire the helper T-cell dependent characteristics of the carrier protein CRM197, purposely presented to the human or animal host's immune system via the nanostructured vector Lipid A-SAEP complex (Endotoxoid A) for mimicking the shape and the size-characteristics of a viral nanoparticle.

The nanostructured vector system of the invention may also include known immunological adjuvants. Such immunological adjuvants may be adjuvants of mineral origin such as alum and/or aluminum salts, such as aluminum hydroxide/phosphate and potassium aluminum sulfate, as well as adjuvants of biological nature that are in use in modern vaccinology.

According to an alternative embodiment the nanostructured vector system according to the invention may comprise a hybrid molecular construct (also named hybrid antigen hereinafter) comprising a helper T-cell dependent carrier protein carrying one or more viral antigens belonging to SARS-Cov-2 virus.

These hybrid antigens may be produced by chemically reacting the components or by using recombinant DNA technology.

According to a preferred embodiment of the present invention said hybrid antigen comprises CRM197 covalently bound to the biologically functional subunit of a viral protein, the Receptor Binding Domain in trimeric pre-fusion state (RBDt), part of the S1 subunit in the Spike glycoprotein of SARS-CoV-2 virus. The target of this molecular construct is the achievement of a hybrid, multi-potent, nanostructured antigen capable of expressing an immunological booster activity against the trimeric RBD (Receptor Binding Domain in trimeric conformation, RBDt, as pre-fusion structure present in the subunit S1 of the S-protein) since its first injection into a mammalian host already primed by a protein antigen (e.g.: Diphtheria Toxoid) cross-reactive with the protein antigen which is part of the structure of such hybrid antigen (e.g.: CRM197) and works as helper T-cell dependent carrier.

According to this fundamental property of the immune system, the preparation and the immunogenic properties of the hybrid conjugate antigen CRM197-RBDt are disclosed. This conjugate is able to immediately boost the DT-primed immune system of a mammalian just following the first dose of it as a prototype vaccine, a still unmet characteristic for achieving anti-CoV-2 immunity as early as possible. In fact, as learned from the clinical experience on the COVID-19 pathology and from the first generation of mRNA vaccines introduced, the first vaccine dose elicits modest levels of pro-inflammatory cytokines (e.g. interferon isotypes) which take some days to peak, preparing the immune system to the quick response of the second dose which, in a few days, elicits an immediate and sharp release of interferon paralleled by a logarithmic increase of IgG isotype antibodies specific for the spike glycoprotein of the virus. Both, post-first and post-second dose interferon signatures have been reported to be associated with the development of the specific IgG antibody responses. Such distinct interferon response phenotypes were also observed in patients with COVID-19 and associated with severity of the pathology, finally associated with differences in the duration of the intensive care.

Accordingly, the hybrid CRM197-RBDt antigen disclosed here as a preferred embodiment of the invention, intends to provide elimination of this gap-time in the window of the immune response by making use of a designed antigen with built-in helper T-cell dependent characteristics, for which the host's background immunological memory is already present, incorporated into the nanostructured Endotoxoid A which, as a nanoparticle system, provides an optimal antigenic presentation to the host's immune system by mimicking the surface of a viral particle. The present invention further provides a production process of the nanostructured vector system incorporating the hybrid antigen comprising the following steps:
(1) reacting under appropriate reductive and then oxidative conditions the helper T-cell dependent carrier protein CRM197 with the viral antigen RBD in order to form covalent, interchain, S—S cross-bridging bonds according to the following stoichiometry of reaction:

3 CRM197(—SH)₂+2 RBDt (—SH)₃

to produce and hybrid antigen having formula CRM1197₃RBD₂;
(2) incorporating the hybrid antigen obtained in step 1) into the Lipid A moiety of Endotoxoid A, resulting in the formation of nanoparticles.

The invention further relates to the hybrid antigen thus obtained having formula CRM197₃RBD₂ and sub-multiples or multiples thereof. As these products are molecular polydispersions, the term multiples referred to the hybrid antigen is intended to cover multiple index of the representative compound (CRM197₃RBD₂)ₙ.

The invention further relates to the nanostructured vector system above disclosed for use in medical field as a vaccine for inducing an immune response when administered to a mammalian host (including a human host or a veterinary).

It is a further object of the present invention a vaccine comprising the nanostructured vector system comprising a complex Lipid A-SAEP or a Lipid A derivative-SAEP derivative (Endotoxoid A) that incorporates a helper T-cell dependent carrier protein for use in the vaccination of a mammalian host against viral and/or bacterial infections, optionally together with other pharmaceutically acceptable adjuvants.

According to the present knowledge of the immune system, the administration of the vaccine to a host will modify one or more specific targets in the immune system of such a host with the subsequent emergence of an immune response.

Vaccines may be made with this vector system by combining the vector system with one or more diverse species specific conformationally-dependent viral antigen(s) such as the envelope Glycoprotein E homodimer that expresses conformational (quaternary) epitopes or a part of an epitope as a vaccine for the Dengue 2 virus; or the viral antigen is the stabilized RSV perfusion F glycoprotein, or its antigenic conformational site phi containing the RSF trimer for Respiratory Syncytial Virus (RSV); or the viral agent is the outer domain epitope of the Glycoprotein gp120, in its native conformation, binding to human broadly neutralizing antibodies as a vaccine for HIV. Other viral conformationally-dependent antigens include at least one of the multiple antigens of the influenza virus (HA, NA, NP and M1) in their respective native conformations, as a vaccine for influenza virus (IV); or the viral antigen for rabies is the rabies glycoprotein trimer RABV-G in a pre-fusion conformational state expressing a functional quaternary epitope, as a vaccine for Rabies Virus (RV); or the viral antigen is the glycoprotein trimer EBOV in a pre-fusion conformational state expressing a functional epitope for Ebola Virus) (EBOV). These vaccines may incorporate the viral agents in conjugated or unconjugated form.

If the vaccine is for a species specific bacteria the antigens may comprise capsular polysaccharides or lipopolysaccharides in conjugated or unconjugated form.

Vaccines may also be prepared using the vector system for species-specific antigens like capsular polysaccharides and/or lipopolysaccharides such as those of the genus Escherichia (e.g. E. coli), Salmonella (e.g.: S. typhi and S. typhimurium), Shigella (e.g.: Shigella flexneri and Shigella sonnei), Klebsiella (e.g.: K. pneumoniae), Pseudomonas (e.g.: Pseudomonas aeruginosa), Acinetobacter (e.g.: A. baummannii), Clostridioides (e.g.: Clostridium difficile) and Staphylococcus (e.g.: Staphylococcus aureus) in a monovalent or polyvalent formulation for the prevention of multiple drug resistant pathogens.

Multiple vector systems may be prepared to make broad spectrum vaccines having a plurality of species specific antigens. These vaccines may be formulated with glycoconjugate-based vaccines (such as Menveo®, GSK or Prevnar®, Pfizer) for making a broad spectrum species specific vaccine for Gram-negative and/or Gram positive bacteria.

The vaccine of the present invention comprising the nanostructured vector system may be formulated for parenteral (i.m, s.c., i.p.) and topical administration (i.n.) in a mammal host.

According to a preferred embodiment the VS-based vaccine according to the invention is administered in an effective amount in the range 0.1-100 μg, preferably 1-50 μg.

The invention also includes a method for the preparation of a vector system which comprises combining a Lipid A-SAEP or a Lipid A derivative-SAEP (Endotoxoid A) with a helper T-cell dependent carrier protein and an immunogenic quantity of bacterial and/or viral antigens. Said bacterial and/or viral antigens may be conjugated and/or unconjugated to said helper T cell dependent carrier protein.

The invention further relates to a method for the preparation of a vector system which comprises combining a Lipid A-SAEP or a Lipid A derivative-SAEP with a conjugated hybrid antigens comprising a CRM197 protein and the viral SARS CoV-2-S-protein or the part of said protein known as RBD (Receptor Binding Domain) expressed by any of the SARS CoV Variants of Concern (VOC) in either a monomeric or a trimeric conformation.

The conjugated hybrid antigens may be formulated with an adjuvant as a species specific vaccine.

The hybrid antigens may be formulated as a species specific vaccine for the prevention of COVID-19 which is based on a combination of the vector system comprising CRM197 and the SARS CoV-2-S protein or the RBD expressed by anyone of the SARS CoV-2 VOC in monomeric or trimeric conformations.

The present invention will now be described for illustrative but non-limiting purposes, according to a preferred embodiment with particular reference to the attached Figures, in which:

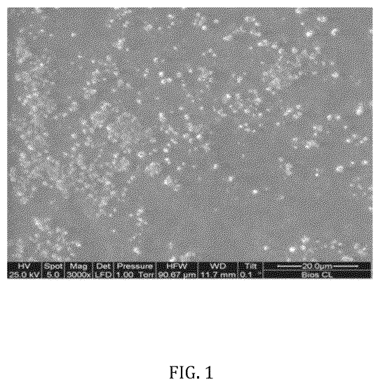

FIG. 1 is an environmental electron microscopy analysis of Endotoxoid A originating from N. meningitidis.

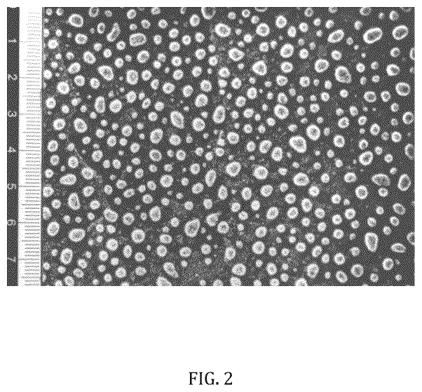

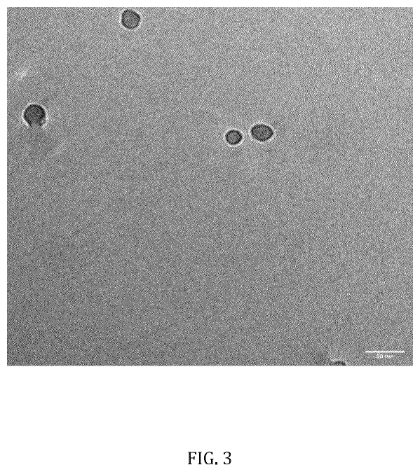

FIG. 2 is a quick freeze, deep etch rotary-replication transmission EM of an Endotoxoid A.

FIG. 3 is a cryo-TEM of the Vector System applied to Endotoxoid A.

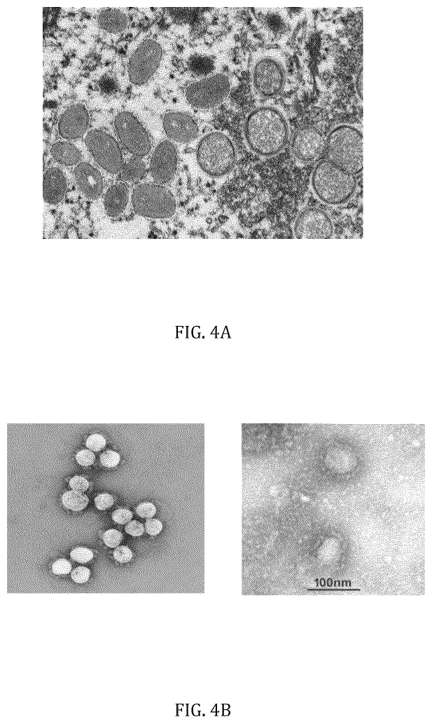

FIG. 4A is an electron microscopic view of the smallpox virus.

FIG. 4B is an electron microscopic views of two different SARS CoV-2 viruses.

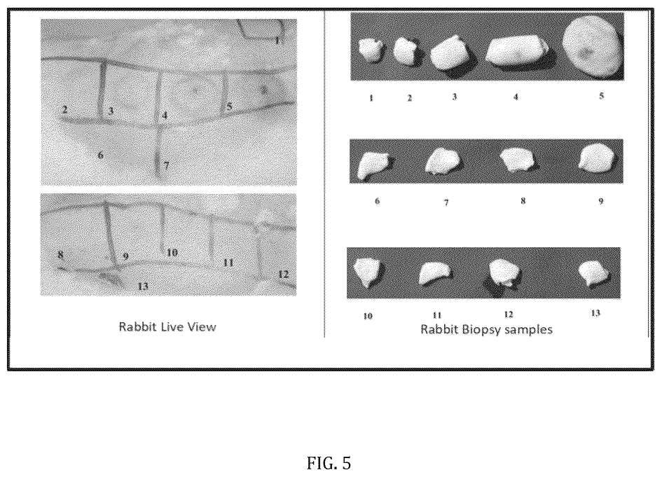

FIG. 5 is a view of lipid A-induced TNFα and IFN-γ mediated, haemorrhagic dermonecrosis.

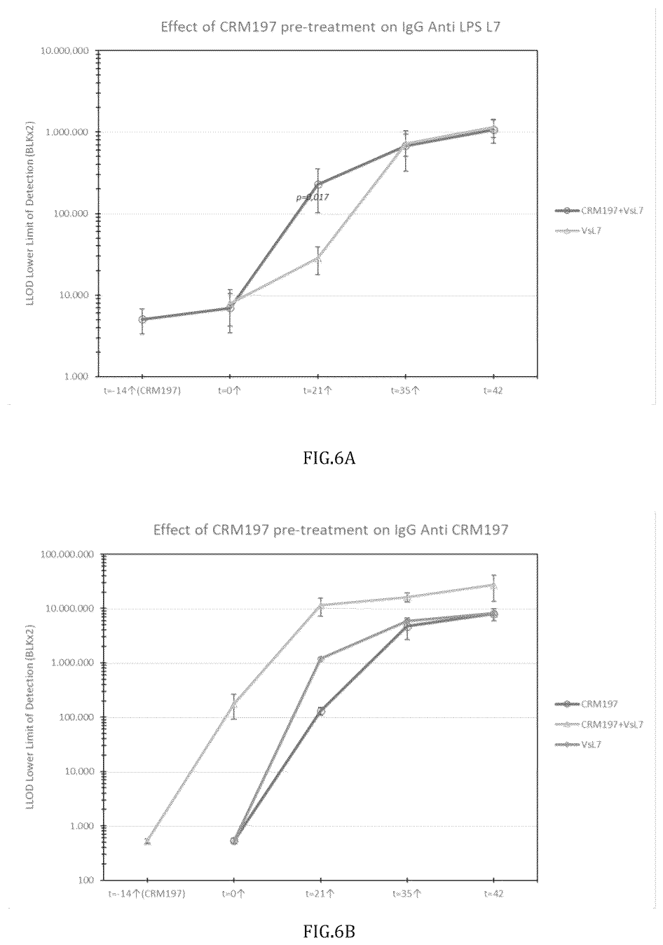

FIG. 6A demonstrates the effect of CRM1 pre-treatment on IgG Anti LPS L7.

FIG. 6B demonstrates the effect of CRM197 pre-treatment on IgG Anti CRM197.

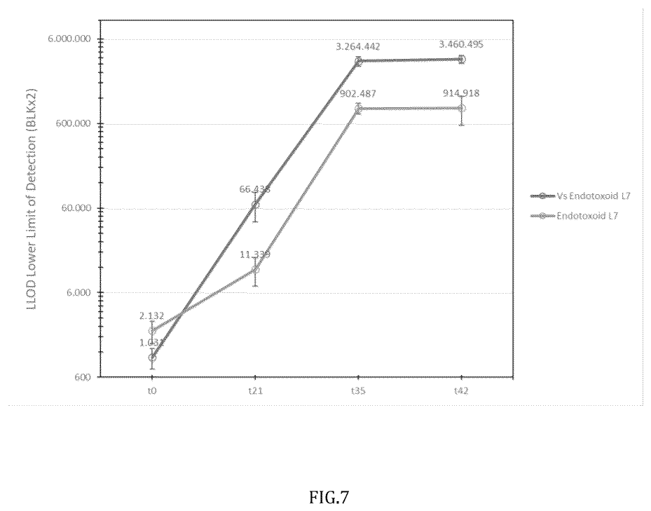

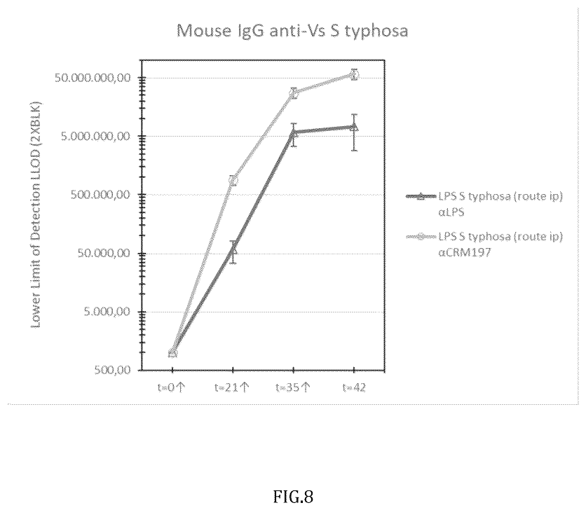

FIG. 7 provides an immunological comparison between Endotoxoid L7 and VS Endotoxoid L7.

FIG. 8 shows the parallel kinetic of induced IgG isotype antibodies to VS components and LPS by S. typhi.

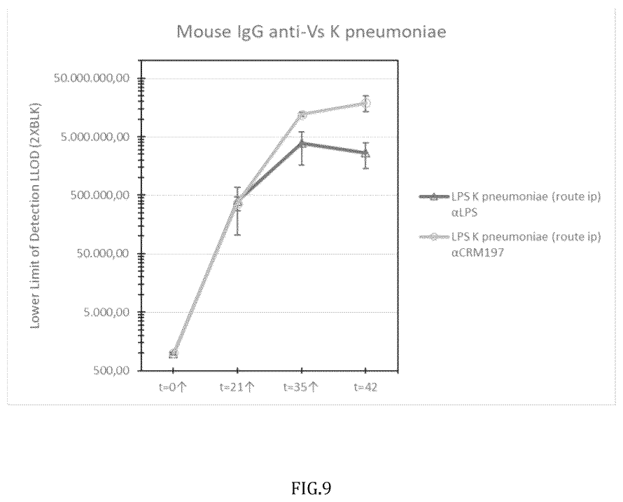

FIG. 9 shows the parallel kinetic of induced IgG antibody to the VS components CRM197 and LPS by type 1 VS K. pneumoniae.

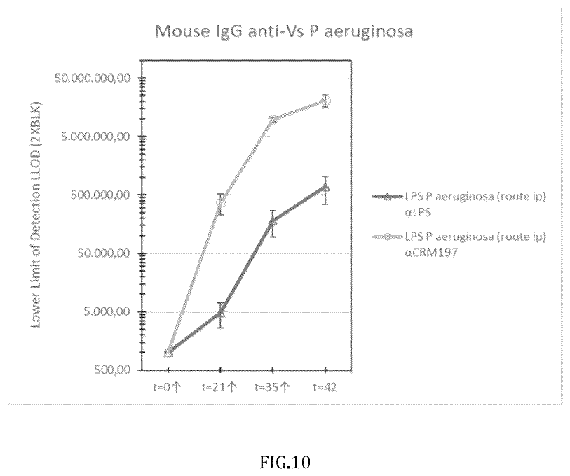

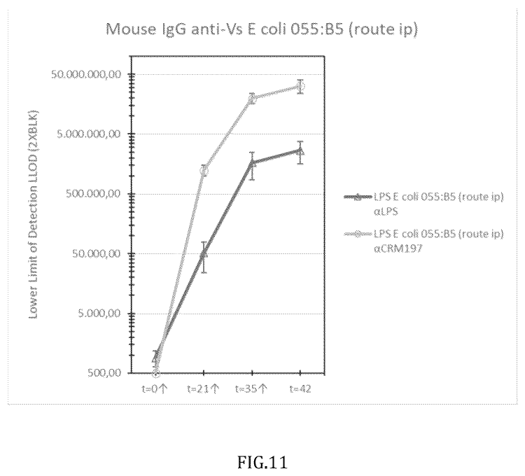

FIG. 10 shows the parallel kinetic of induced IgG isotype antibody to the VS components CRM197 and LPS by VS P. aeruginosa 111.

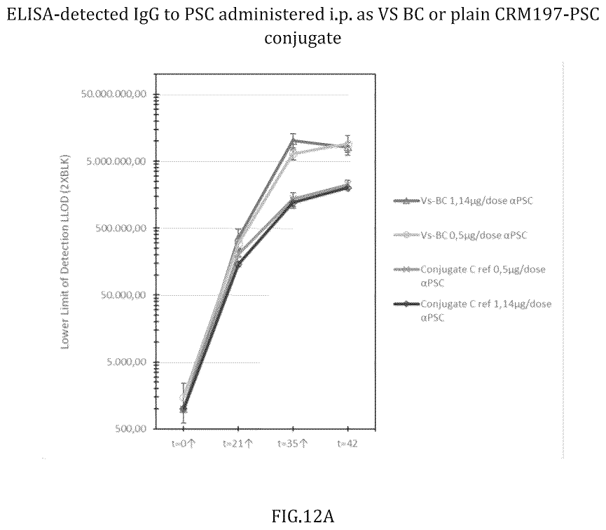

FIG. 11 shows the parallel kinetic of induced IgG isotype antibody to the VS components CRM197 and LPS by VS E. coli.

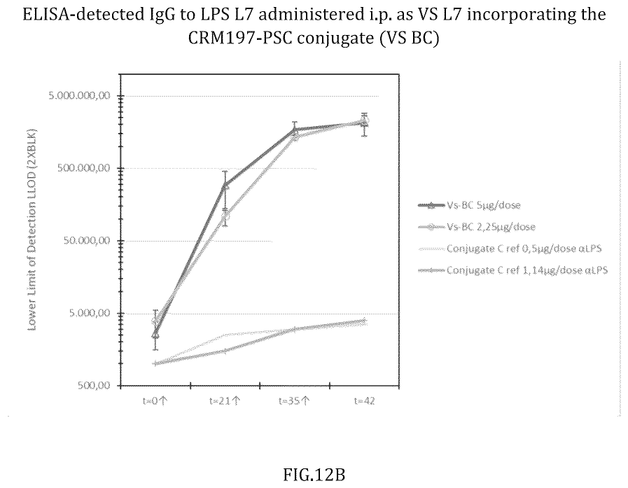

FIGS. 12A and 12B show the immunogenicity of the conjugate CRM-Polysaccharide Group C when it is vectorized by VS N. meningitidis L7 and administered i.p.

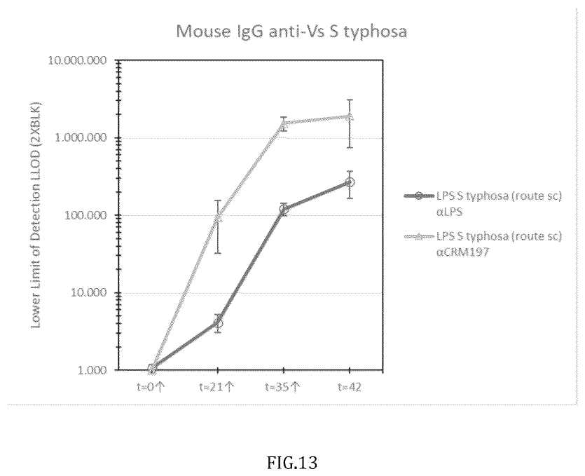

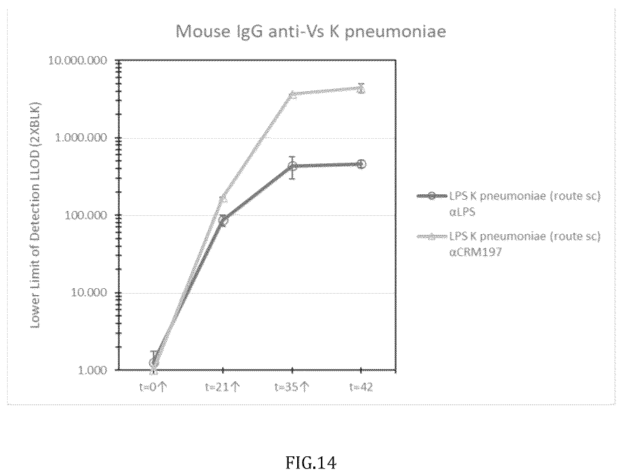

FIGS. 13, 14, 15 report the results of the s.c administration to mice of several species specific mouse IgG anti-VS.

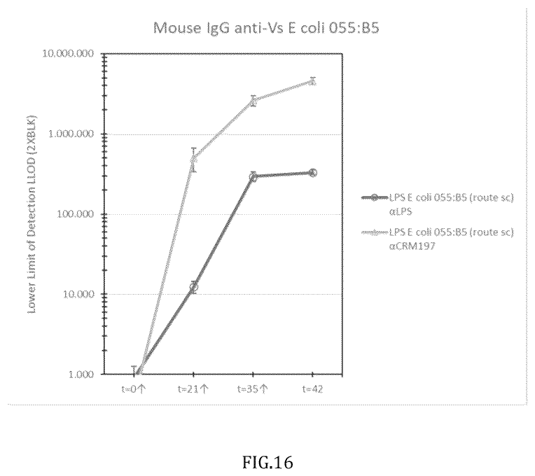

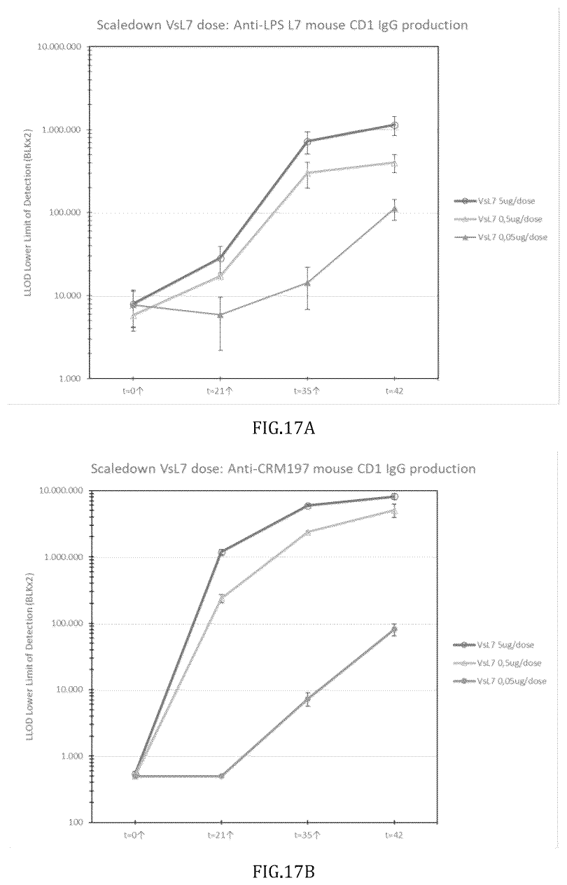

FIG. 16 shows the results of s.c. administration to mice of mouse IgG anti-VS E. coli.

FIG. 17A shows the results of a determination of the minimum immunogenic dose for VS N. meningitidis L7.

FIG. 17B shows the results of a scaledown VS L7 does on anti-CRM197 mouse CD1 IgG production.

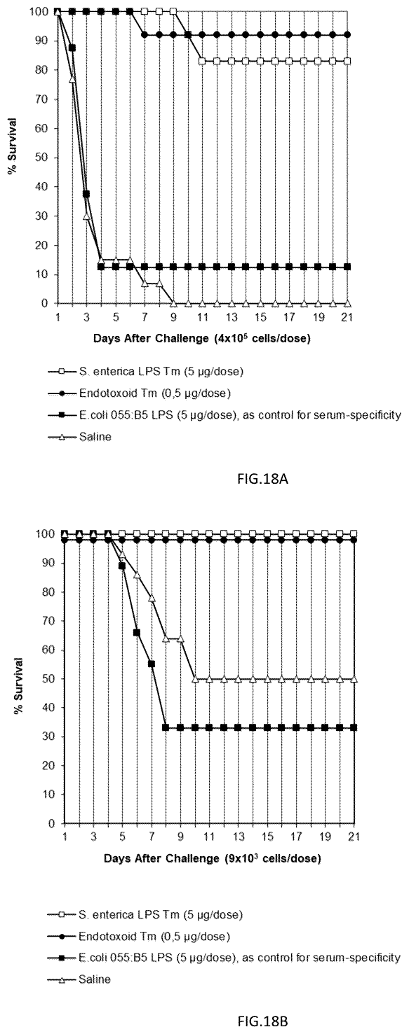

FIGS. 18A and 18B show the active protection provided by VS Endotoxoid from S. typhimurium LPS against lethal infection by a homologous strain.

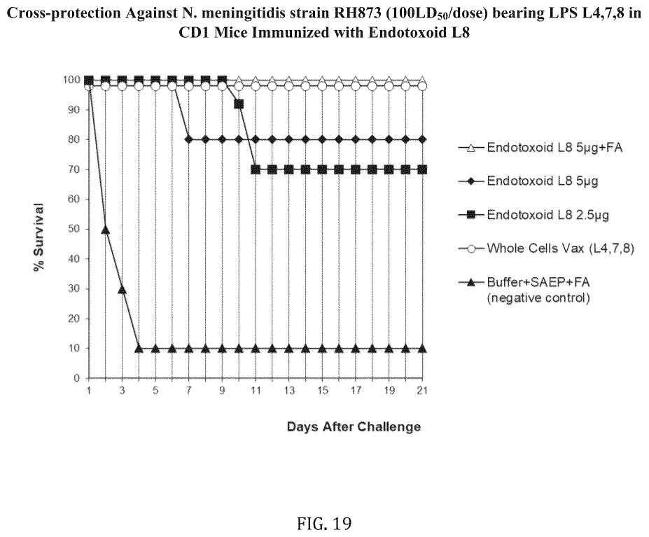

FIG. 19 demonstrates the active cross-protection of a VS Endotoxoid L8 strain against a N. meningitidis strain.

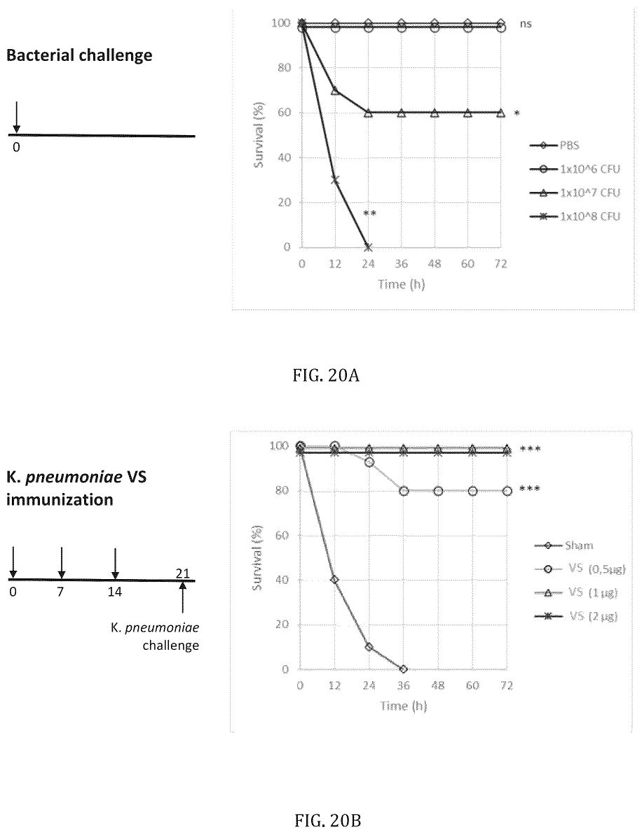

FIGS. 20A and 20B demonstrate the protective effect of VS K. pneumoniae type 1 in CD 1 mice against bacterial challenge by a homologous K. pneumoniae strain.

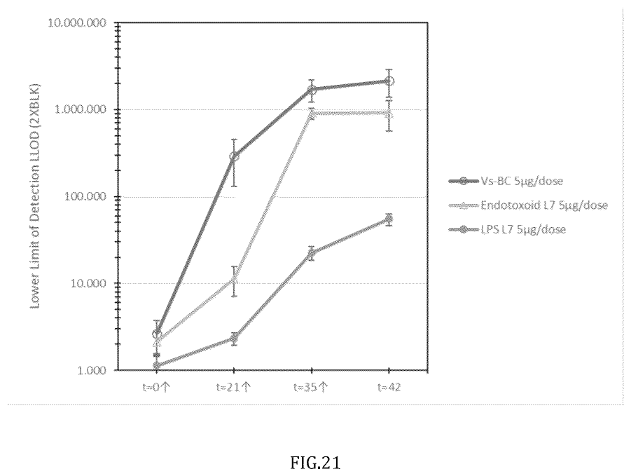

FIG. 21 demonstrates the relative immunogenicity of the VS components induced by the VS of N. meningitidis or the homologous Endotoxoid in comparison to native LPS.

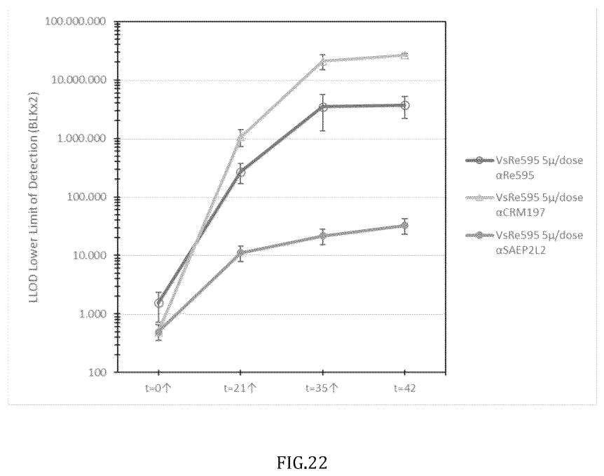

FIG. 22 reports the relative immunogenicity of the VS components induced by the VS formulation relative to LPS Re 595 from Salmonella minnesota.

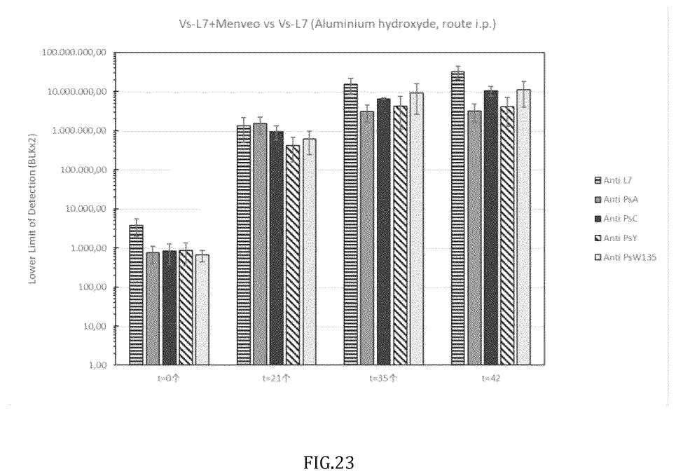

FIG. 23 provides a comparison of IgG titers of anti-L7 versus anti-Group A, C, Y and W.

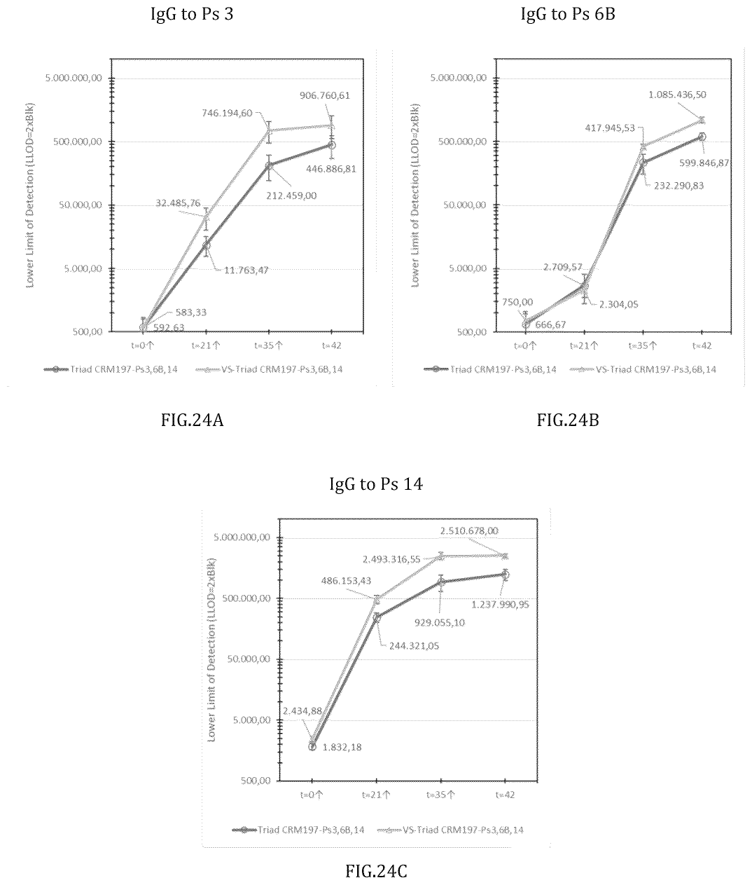

FIGS. 24A, B and C compare the immunogenic activity of the VS that incorporates the Triad multivalent conjugate antigen CRM197-PS 3,6 B,14 to the immunogenic activity itself.

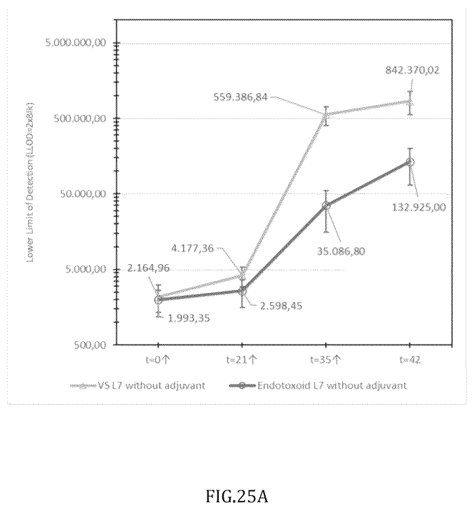

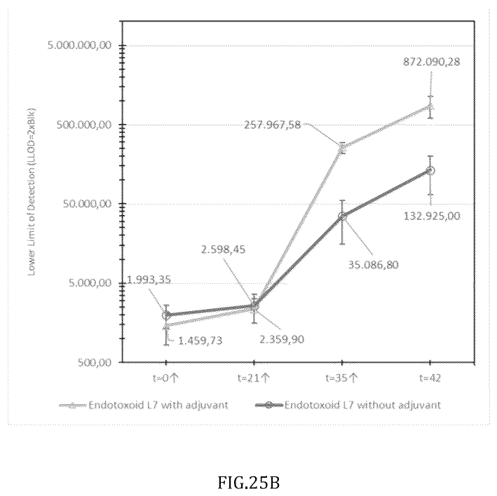

FIGS. 25A and B provide a comparison of the immunogenic properties of N. meningitidis LPS L7 in Endotoxoid vs. VS formulation.

The following non-limiting examples are now provided for a better illustration of the invention, in which several species-specific nanostructured vector systems (VS) and hybrid antigens of the invention were produced and their immunogenic and protective properties in animal models were assayed.

### EXAMPLES

### Example 1: Preparation of the Conjugate Hybrid Antigen and Related Nanostructured VS

The production process of the nanostructured VS conjugate comprising the hybrid antigen of the invention involves two main steps:
(1) preparation of the hybrid antigen CRM197₃RBD₂ based on the formation of covalent, interchain, S—S cross-bridging bonds, and
(2) incorporation of the hybrid antigen into the Lipid A moiety of Endotoxoid A, resulting in the formation of nanoparticles.

The RBDt as part of the S Protein (AA sequence 319-541 not containing the poly-histidine fusion tag at its C-terminus) has about 10³ times the affinity to an ACE2 receptor than its monomeric form but is a weak antigen that may take significant benefit from the carrier effect of CRM197, even after a single injection of a specific host, because of the “booster” activity of CRM197 on the IgG isotype antibody populations primed by Diphtheria Toxoid (present in the “universal” DTP paediatric vaccine).

RBD (as Trimer in its pre-fusion conformation) contains 3 unpaired Cys residues, as source of —SH groups (reduced form), localized at position 538 of the primary sequence (AA 319-541) of each of the three monomers, therefore outside of the sequence binding to the ACE2 receptor (AA 438-506). The remaining 4 pairs of Cys-Cys residues (oxidized form) contained in each of the three monomeric sequences are localized at positions 336,361,379,392,432,480,488,525. CRM197 features 1 pair of Cys residues (easily reducible) which allows the two structural fragments A (193 AA) and B (342 AA) to be covalently linked (bridging) even when the primary sequence of CRM197 (535 AA) undergoes limited proteolysis in the Arginine-rich AA sequence encompassing the two Cys residues (AA sequence 185-201).

According to the above, before the purified RBDt (expressed in a convenient cell line) and the purified CRM197 are stoichiometrically mixed in consideration of the amount of the Cys-SH residues actually available in both proteins (2-SH/mole for CRM197 and 3-SH/mole for RBDt), the CRM197 solution (aqueous buffer solution at pH 6.0-7.0 containing ionic strength equivalent to 0.145 M NaCl) is treated with a very low concentration of reducing agent (range 10-3 to 10-4 M) in very mild conditions (1-2 hours at room temperature) in order to promote the reducing “scavenger” activity on the available free —SH groups. The most useful reducing agents for this purpose are β-mercaptoethanol (ME) or 1,4-dithio-d-threitol (DTT) or tris(2-carboxyethyl)phosphine (TCEP), the latter being chemically stable, non volatile and odorless. Once the solutions of the two proteins are mixed, the resulting solution is kept at room temperature for 1 hour. ¹H-NMR and CD and other spectroscopic methods can be purposely used to control the quantitative number of reduced —SH which are available in the resulting solution with respect to the designed stoichiometry.

The RBDt (pre-fusion state of the Trimer) is composed of 3×223=669 AA (MW=76.9 K) while CRM197 is composed of 535 AA (MW=61.5 K). Because of the number of easily reducible Cys residues in each molecular entity, one may rationalize that two (2) molecules of RBD Trimer (total of 6 —SH groups) would “cross-bridge” with up to three molecules of CRM197 (total of 6-SH groups) forming a new conjugate entity CRM197₃RBD₂ theoretically composed of 2,943 AA and with a calculated (approximate) MW=338.5 K. Multiples and/or submultiples of the indicated integers may be present in the resulting molecular populations, as entities composed of a different stoichiometry being part of a Gaussian-like distribution of MW.

Following the incubation, Molecular TUF (Tangential Ultra Filtration) is used with a convenient LMW size-exclusion membrane (membrane cut-off in the MW range 1 OK-100K), in order to eliminate any trace amount of the reducing agent used as “scavenger” and any eventual presence of unreacted antigens, which can be recovered for a further reaction cycle. Oxidation then follows by an oxidizing agent (e.g.: diluted Hydrogen Peroxide solution 10-3 M or diluted Iodine solution 10-3 M, either one with pH adjusted in the range 7.0-7.5) so that the final molar excess is 10² to 10³ times the amount of the —SH groups present in solution, in order to allow the formation of the new covalent S—S bridges between CRM197 and the RBD Trimer yielding a hybrid polyvalent antigen (cross-bridged protein) with the theoretical composition CRM197₃RBD₂. The whole preparation process takes few hours (6 to 8 hours), with the main parameter to keep under control being the accurate molar stoichiometry of the role played by the conjugate, as related to the 6 pairs of —SH groups resulting in the corresponding 6 pairs of —S—S— cross-bridged groups after oxidation.

The illustration set forth, below, is provided as an example to explain the theoretical symmetric geometry of the hybrid antigen CRM197₃RBDt₂ based on the calculated stoichiometry of the reacting Cys-SH residues involved in the cross-bridging coupling reaction:

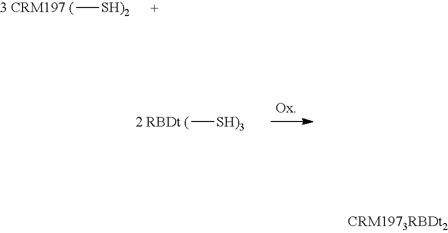

As related to the RBDt structure, this is an adaptable entity that can be considered as a single cross-reactive structure among the variants of concern for CoV-2 (expressing Type 1 antigenicity) or considering a mixture of RBDt structures of different variants in case of non-complete cross-reactivity or Type 2 antigenicity (determinant sharing) among such variants (e.g.: Delta variant vs. Omicron variant).

Eventually, different RBD sequences from different viral variants of concern (VOC) can be used for a broad-spectrum multivalent vaccine composition and formulation. Also, an alternative strategy for producing the polyvalent antigen may include the full sequence (S1+S2) of the Spike glycoprotein which contains additional epitopes to the RBD sequence (e.g.: the NTD sequence in S1 and the fusion machinery stem helix sequence in S2) which are well represented within the human IgG antibody response to CoV-2 while paralleled by the specific T-cell immune response. If preferred, the whole structure of the S Proteins (S1+S2) may be used in replacement of RBDt, for expression of the most comprehensive universe of anti-S Protein epitopes resulting in the T-cell immune response paralleled by the induction of virus-neutralizing and virus-non-neutralizing antibodies (the latter, likely having importance in preventing the endocytotic mechanism of the virus entry in host cells, thus complementing the neutralizing immune response to the RBDt).

In this case, the chemistry involved for covalently linking CRM197 to the full sequence of the Spike glycoprotein can be the one involving the succinimidyl ester derivatives (BiosYnth's U.S. Pat. No. 10,300,135 B2) or any other protein reagent useful for cross-linking the two protein entities.

The resulting cross-linked molecular entity (CRM197Protein-CO—NH-ProteinSpike) may have, however, a different stoichiometric composition with respect to the cross-bridged entity (CRM197Protein-S—S-ProteinRBDt) above described. In fact, while the mean MW of the purified hybrid antigen CRM197₃RBDt₂ has been found to be 340 K (±15 K or ca. ±5%) by MALDI-TOF and GPC calibrated by Pullulan (therefore in the range value theoretically calculated) a cross-linked conjugate CRM197Protein-CO—NH-Protein Spike may reach multiples of such a value because of the multifunctionality of both components of the conjugate. Whichever hybrid antigen is selected, the resulting purified multi-potent nanostructure is an immunocompetent antigen that expresses multiple epitopes for RBDt and CRM197.

The above described hybrid antigen (CRM197)₃-(RBDt)₂ and the related CRM197-S1/S2 Spike protein antigens made by chemical synthesis, can be also obtained by recombinant DNA techniques given the fact that all the genetic sequences of CRM197 and RBD/Spike protein are well known and identified as translated amino acid sequences with their amino acids in unique positions (see above). It is therefore feasible making genetic molecular constructs of DNA/RNA probes leading to the translated synthesis resulting in the genetic sequence of CRM197 fused with that of RBD or with that of the whole S1/S2 Spike protein.

### Example 2: The Vector System Composed by Endotoxoid A and CRM97-RBDt

Endotoxoid A is the molecular base for a safe and powerful helper T-cell dependent vector for different protein antigens, while RBD (as a monomeric entity or as pre-fusion trimeric conformers) is a weak antigen when isolated from the full-size natural spike glycoprotein. Accordingly, RBD takes significant benefit from the carrier effect of CRM197 as well as from that of the Endotoxoid A vector which is able to incorporate a variety of protein structures in the form of hydrophobic complexes yielding immunogenic nanostructures. Different RBDt sequences from different viral variants (VOCs) can be used for incorporation in a broad-spectrum multivalent vaccine composition and final product formulation. For an optimal “booster” activity since the first dose, RBDt is incorporated into the Endotoxoid A nanostructure together with CRM197, thus taking advantage of the existing anti-DT immunity in the host. In particular, the new developed hybrid antigen CRM197₃RBD₂ can be incorporated into the Endotoxoid A nanostructure directly, therefore by replacing the unconjugated CRM197 component, offering a new strategy for vectorizing subunit-based viral vaccines.

Incorporation of the CRM197-based hybrid antigen into the Endotoxoid A Vector System

Lipid A is a TLR-4 agonist and in purified form (A. Rustici et al., Science 259:361-365 (1993) is a molecular entity in micellar status that is obtained by chemical hydrolysis of purified R-type or R/S-type LPS, or it can be directly obtained by bacterial fermentation of the Gram-negative mutant Salmonella minnesota Re 595, whose structure has been assessed by ¹H-NMR spectrometry and here below reported:

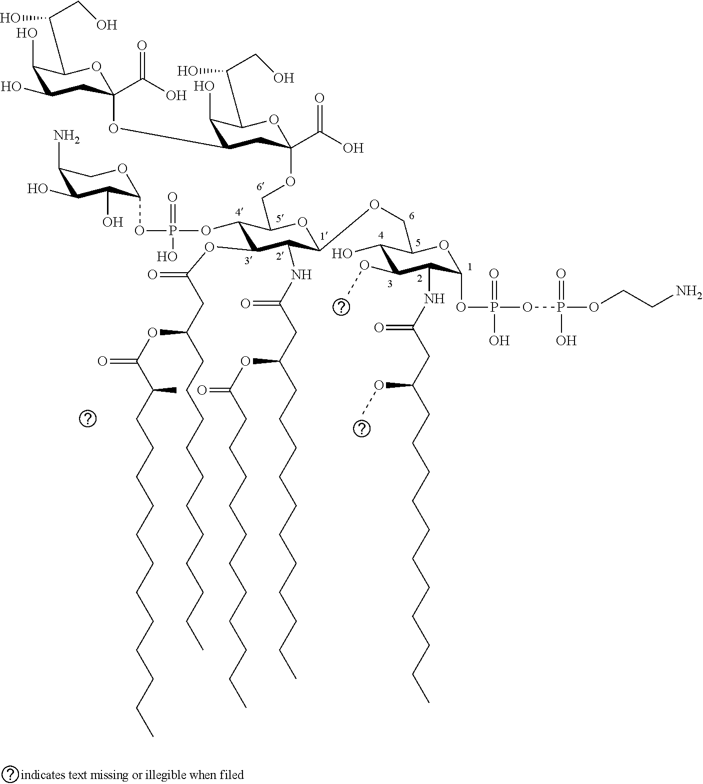

LPS Re 595 is here considered a Lipid A derivative like other chemotype LPS (e.g.: Re,Rc,Rs structures).

The Lipid A component, or its R-derivatives and S-derivatives, as part of the vector system can incorporate the purified RBDt or multiple variants of the RBD trimer or the conjugate CRM197₃RBD₂ (hybrid antigen) forming a soluble hydrophobic complex, stabilized by strong hydrophobic forces, in aqueous solution.

SAEP (a category of TLR-4 antagonists) are structures similar to the drug-category of polymyxins (e.g.: PmB) containing the amphipathic characteristics similar to those reported for SAEP-2 and its structural derivatives (A. Rustici et al., Science 259: 361-365, 1993) with comparable physical-chemical characteristics but with no expression of biological toxicity because they are constituted by natural amino acids unlike the Polymyxins drug-category with the (ABn/ABCn motif and the ratio Cationic/Hydrophobic amino acids ≥0.5).

SAEP or a SAEP derivative (whose general structural features may be determined by ¹H/¹³C-NMR spectrometry) is then added to the solution containing the Lipid A, with the hybrid antigen, in order to form the high affinity stoichiometric complex Endotoxoid A, which incorporates the hybrid antigen while precipitating.

Following the preparation of the hybrid antigen, this is incorporated within the micelles of Lipid A (T=37° C.×1 hour) by simply mixing up the antigen and the Lipid A at a ponderal ratio in the range 0.8-1.2 (w/w), at 37° C. and in aqueous solution, without any ionic strength and adjusting the pH in the range 6.5-7.5. Finally, the Lipid A-selective SAE Peptide (e.g.: SAEP2 in cyclic conformation or an equivalent peptide structure with similar physical chemical characteristics) is added at the ponderal ratio 1.0 (w/w) with Lipid A (at r.t.). The immediately formed Endotoxoid A, which incorporates the hybrid antigen into the micelles, precipitates in the form of a nanostructure. The hybrid antigen is then extensively washed with excess of saline solution (pH=7.0) by TUF (Tangential Ultra Filtration) and processed by physical-chemical, immunochemical and immunological analysis.

The stoichiometric characteristics of the nanostructured hybrid antigen, predicted to be:

Lipid A-(CRM197₃RBDt₂)-SAEP=1:1:1(w/w)

with a variability of the ratio(s) within 25% of the reported values, have eventually fallen within the range-value predicted on the basis of the calculated stoichiometry. Endotoxoid A is therefore the detoxified entity resulting from the binding of SAEP to Lipid A and, by incorporation of CRM197 (in this case as part of the hybrid antigen CRM197—RBDt), it becomes the new molecular entity here named Vector System which assumes a nanostructured conformation here shown in FIGS. 1 and 2 which are micrographs resolved by different techniques of Electron Microscopy (EM). The VS is very stable in a broad range of temperatures and can be stored as a liquid at 4-8° C. with no need to employ low or ultra-low conservation temperatures.

Because of the specific principle on which the VS is based, the protein antigens present in the universal paediatric plan of immunization which considers the DPT (Diphtheria-Tetanus-Pertussis) background immunization as implemented by Country-specific and International Health Services, the carrier proteins useful for being incorporated into the VS are the DPT-related antigens including the DT cross-reactive protein CRM197 so that Tetanus Toxoid or Pertussis Toxoid or Diphtheria Toxoid may serve for the purpose in replacing CRM197. Of course, the stoichiometric properties defining the composition of the VS, as related to the components, may change in relation to the different structure and molecular weight of such components. The micelle-like nanostructure of Endotoxoid A is well visible and resolved in the conditions of ES-Electron Microscopy (ESEM) at a magnification range 3,000-7,000× as well as in Transmission Electron Microscopy (TEM) at higher magnification shown in FIGS. 1 and 2.

FIG. 1 is an ESEM analysis of the Endotoxoid A originating from N. meningitidis Group B. Micrograph shows the actual micellar conformation of the molecular entity in its native, aqueous, environment. Magnification 3,000×. Bar indicate size-dimension. Microscope Philips Mod. XL30 TMP operating at the pressure of 1.0 Torr and using a Peltier cell at the T=15° C. FIG. 2 is a Quick-freeze, deep-etch, rotary-replication Transmission EM of the Endotoxoid A originating from N. meningitidis Group A made by negative staining with ammonium molybdate. The sample was mounted at 50 g/ml and printed at high magnification (108,000×; 1 cm=92 nm) in a Thermo Fisher Scientific Tecnai G2 Spirit microscope.

The nanostructure characterizing the Vector System is here below represented in FIG. 3 as shown by the Cryo-TEM technique. The Cryo-TEM of the Vector System was applied to Endotoxoid A that incorporated the Helper T-cell dependent protein CRM197. Sample mounted at 0.2 mg/ml in a Philips CM200FEG microscope (bar=50 nm). The mean size of the micelles are <50 nm thus fitting in the IUPAC definition as nanoparticles (1-100 nm). In order to compare the dimensions of the of the SARS CoV-2 virus with the dimensions of a virus like the smallpox virus by EM (ca. 200 nm) as reported in Science, vol. 378:902-903 (2022), FIGS. 4A and B are provided to describe the respective dimensions of these viruses.

The size of SARS CoV-2 virus is ca. 50 nm according to several different sources in the current literature, therefore just comparable to the Endotoxoid A component and the derived Vector System disclosed in the present application.

### Example 3: Safety of Endotoxoid A as a Component of the Vector System (“In Vitro” Vs “In Vivo” Studies)

The Lipid A-SAEP complex (Endotoxoid A) has been extensively investigated in the past activities of BiosYnth S.r.l. in animal models as well as in humans and the LAL assay has been found a good and simple probing test for evaluating the absence of pyrogenicity together with absence of release of key pro-inflammatory citokine-mediated toxicity (TNFα, I1-6, IFNγ) in animal models and in a pivotal Phase I clinical trial (adult volunteers 18-45 ys). Accordingly, a LAL-detected reduction activity in the range 2.7/3.0 log (500-1,000 times) with respect to the activity of native Lipid A/LPS can be considered a safe evaluation parameter for the purpose. The routine results obtained with various Endotoxoids are reported below in Table 1 are consistent with such operational parameter evaluating “in vitro” the detoxification rate of specie specific Endotoxoids. The identified detoxification rate by LAL “in vitro” test did correlate with the “in vivo” test showing the lack of release of the serum detectable pro-inflammatory cytokine TNFα which is the cytokine involved in the first step of the cytokine cascade leading to the toxic effects of Lipid A, quite structurally conserved within the interspecies structures of LPS. Another animal model for assessing the safety of the Endotoxoid component of the VS is the rabbit. Table 1 discloses the quantity of TNF released in the serum of CD1 mice following a single s.c. injection of a specie specific LPS and the homologous Endotoxoid. TNF was titered in the serum of each mouse, 90 min. from injection, by bioassay. Data are expressed as mean of 5 mice/group. Endotoxoids were previously tested by LAL assay for determining the detoxification rate with respect to the homologous LPS. LAL-detected detoxification rate of each Endotoxoid was ≥2.7 log (≥99.5%).

TABLE 1

TNF

| | | % |
|---|---|---|
| Antigen (dose) | (ng/ml) | (p-value) |
| LPSL8 N. meningitidisA(0.5 μg)* | 24.5 ± 7.8 | 100 |
| Endotoxoid L8 | 0.1 ± 0.02 | 0.4 |
| | | (p < 0.01) |
| LPS S. typhimurium (5 μg) | 35.2 ± 13.8 | 100 |
| Endotoxoid Tm | 0.5 ± 0.25 | 1.4 |
| | | (p < 0.01) |
| LPS NTHi** (5 μg) | 9.8 ± 4.6 | 100 |
| Endotoxoid NTHi | 0.4 ± 0.1 | 4 |
| | | (p < 0.01) |

*LPS L8 from N. meningitidis Group A (strain A1) has been used at 1/10 amount with respect to the other specie-specific LPS because of its significantly higher toxicity and consequent activity in inducing serum TNF.
**NTHi: Non-typeable Haemophilus influenzae

FIG. 5 shows the well-known “Schwartzman reaction” effect in the skin of the animal, which is due to the Lipid A activity in inducing TNFα and IFNγ as pro-inflammatory cytokines responsible for opening dose-response lesions in the skin. No inflammatory activity is seen for the Endotoxoid A even at high dosages. FIG. 5 displays Lipid A-induced, TNF-α and IFN-γ mediated, haemorrhagic dermonecrosis in the rabbit (Schwartzman Reaction) which is a crucial test for assessing Safety of Endotoxoid-based Vaccines.

The following materials were used to obtain the data displayed in FIG. 5:
1. Saline
2. LOS 2.5 ug
3. LOS 5 ug
4. LOS 25 ug
5. LOS 50 ug
6. Endotoxoid A #1 2.5 ug
7. Endotoxoid A #1 5 ug
8. Endotoxoid A #1 25 ug
9. Endotoxoid A #1 50 ug
10. Endotoxoid A #2 2.5 ug
11. Endotoxoid A #2 5 ug
12. Endotoxoid A #2 25 ug
13. Endotoxoid A #2 50 ug

### Example 4: Formulation of the Subunit Vaccine for SARS CoV-2 in Animals and Humans

It is postulated that the dose-formulation of the hybrid antigen in humans should be comparable to the dosages currently known for vaccines using the CoV-2 Spike glycoprotein and in vaccines using the carrier protein CRM197 (Glycoconjugates for meningococcal and pneumococcal capsular polysaccharides) that is in the range 1-100 μg/dose with the optimal dose in the range 10-50 μg/dose including an effective amount of the selected antigenic material. Optimal doses may be determined by routine experimentation in mammalian, murine and non-human primate models using conventional methods such as immunization and challenge techniques. The vaccine per se may be formulated by dissolving or dispersing the vectored hybrid antigen in water for injection containing salts for buffering the pH at physiological value (within the range 7.2-7.4) and keeping isotony at the value of 0.145 M or any suitable non-reactive liquid or solid diluent at a concentration that will facilitate parenteral administration of a dose that will produce a positive immunologic response.

Because of its high stability in aqueous conditions, the VS may be formulated for parenteral use (i.m., s.c., i.p.) to specifically induce systemic immunity as well as dispersed/nebulized in aerosol/spray solutions for intranasal use (i.n.) to specifically induce local immunity.

For the animal experiments disclosed in the present Application, each antigen incorporated into the VS has been dosed between 1-10 μg for i.p. injection in CD1 female mice in order to follow the immunization kinetic of the IgG antibodies.

### Example 5: Animal Models for Testing the Invention in Preclinical Studies

The direct demonstration, in a murine animal model, that anti-DT immuno-memory does allow the immediate boosting of IgG Abs to the antigen CRM197 incorporated into the Vector System has been obtained in CD1 mice as a significant mammalian model paralleling humans for anti-Diphtheria Toxin antibody-mediated protection according to the test-requirements of the International Pharmacopoeia for the paediatric DPT vaccine. Two groups of 10 animals each of CD1 female mice were injected in parallel with: 1)—CRM197 alone to induce an anti-DT immunological background status mimicking the human anti-DT serological status, and; 2)—the VS based on the antigen LPS L7 which incorporated CRM197 for inducing the priming effect for the LPS L7 antigen. Two weeks later, both groups of mice were injected with the VS containing LPS L7, followed by a third injection of the same antigen three weeks later. IgG antibodies were then quantitated against L7 and CRM197. All injections were made s.c. or i.p. As postulated by the present invention, the murine group primed by CRM197 and containing IgG anti-CRM197 (that is, in humans IgG anti-Diphtheria Toxin/Toxoid IgG) was immediately boosted, by the carrier-induced effect, against the carried LPS L7 by high titers of IgG in contrast to the group of non-primed mice that were boosted only after the second injection of VS as shown in FIG. 6A. As shown in FIG. 6A, the anti-L7 immune response reached an immediate titer of IgG mimicking a booster effect which was ca. 10 times (1 log) higher than that reached by the classic schedule of immunization used in the compared experiment without “priming effect”. At the end of the immunization period both groups of animals reached a comparable level of IgG anti-L7 antibodies, although the “CRM197-primed” group had the expected peak of IgG titers two weeks earlier, due to the Helper T-cell dependent and B-cell dependent “memory effect” that the protein exercised on the host's immune system. Both antigens, CRM197 and Endotoxoid L7, behaved as T-cell and B-cell dependent antigens having comparable kinetics in the IgG-specific sigmoidal curves as shown in FIG. 6B.

The new concept on which the VS is based allows a flexible use and a flexible composition/formulation of the VS as related to the Lipid A structure. It can be a Lipid A or a Lipid A derivative or the Lipid A moiety of a species-specific LPS). In addition, the incorporated Helper T-cell dependent carrier protein, which may be CRM197 or a DT or TT or PT antigen in unconjugated or conjugated form, in the DPT vaccine.

### Example 6: Immunity Induced in a Murine Animal Model by the Species-Specific VS in which Endotoxoid A is Formed Between SAEP and the Lipid A Moiety of the Species-Specific LPS

Each of the exemplified VS reported below, has the following mean composition: 5 μg LPS/2.5 μg SAEP/2.5 μg CRM197 and is prepared according to the procedure previously disclosed in the present application.

[LPS_{(species-specific)}+SAEP]+CRM197→Vector System_{(species-specific)} Endotoxoid A

FIG. 7 provides data derived from an immunological comparison of LPS L7 derived from Neisseria meningitidis (Endotoxoid L7) and the VS Endotoxoid L7 (containing helper-T cell dependent protein CRM197 as a component of the VS). The data in FIG. 7 demonstrates the improved immunogenicity of LPS L7 due to the effect played by the VS in CD1 mice.

FIG. 8 provides data derived from a comparison of the parallel kinetic of induced IgG isotype antibodies from S. typhi to the VS components CRM197 and LPS by the VS S. typhi in CD1 mice.

FIG. 9 provides data derived from a comparison of the parallel kinetic of induced IgG antibodies from K. pneumoniae type 1 to the VS components CRM197 and LPS by VS K. pneumoniae type 1 in CD1 mice.

FIG. 10 provides data derived from a comparison of the parallel kinetic of induced IgG antibodies from Pseudomonas aeruginosa: the parallel kinetic of induced IgG isotype antibody to the VS components CRM197 and LPS by the VS P. aeruginosa 111 in CD1 mice.

FIG. 11. provides data derived from a comparison of the parallel kinetic of induced IgG antibodies from Escherichia coli (055:B5): the parallel kinetic of induced IgG isotype antibody to the VS components CRM197 and LPS by the VS E. coli in CD1 mice.

FIGS. 12A and B show the significantly improved immunogenicity of the conjugate CRM197-Polysaccharide Group C when vectorized by the VS N. meningitidis L7. This formulation allows induction of specific immunogenicity for Meningococcus Group B (L7) and Group C (PsC) as bivalent vaccine (VS BC) in CD1 mice.

In addition to the i.p. immunization route, some of the prepared species-specific VS have when administered by the s.c. route have demonstrated the increased potential of the VS in inducing systemic immunogenicity in CD1 mice as shown in FIGS. 13,14,15,16. In all cases, the i.p. route has shown higher immunogenicity for the VS with respect to the s.c. route, an observation explainable with the direct stimulation of the intestinal Peyers' patches by the antigens.

FIG. 13 provides data that compares the immunogenic response to VS S. typhi (serovar typhosa) made with either LPS or CRM197 when administered s.c.

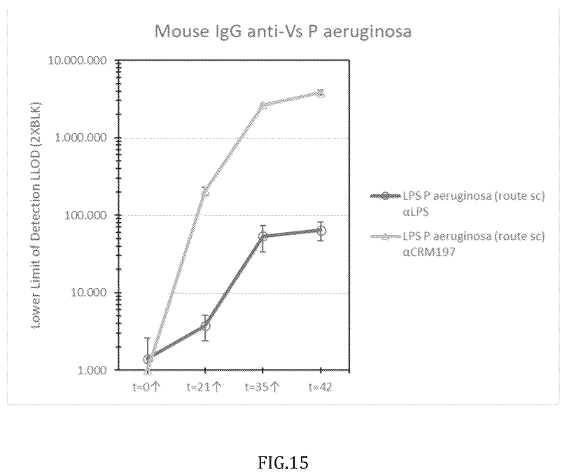

FIG. 14 provides data that compares the immunogenic response to VS K. pneumoniae type 1 made with either LPS or CRM197 when administered s.c.

FIG. 15 provides data that compares the immunogenic response to VS P. aeruginosa made with either LPS or CRM197 when administered s.c.

FIG. 16 provides data that compares the immunogenic response to VS P. coli055:B5 made with either LPS or CRM197 when administered s.c.

FIG. 17 provides data for the Determination of the Minimum Immunogenic Dose (MID) for the VS N. meningitidis L7. The VS is fully immunogenic in CD1 mice at the dose of 50 ng of L7 antigen or lower.

### Example 7: Alternative Route of Administration of Endotoxoid and VS-Based Vaccines

In the above disclosed immunological experiments that were conducted in CD1 mice, the route of administration was i.p. and s.c. For bacterial antigens, these are the classical routes for vaccine use as these routes ensure induction of systemic immunity. In the case of viral antigens these routes are mostly appropriate too, even though for respiratory viruses like SARS CoV-2 a local immunization (e.g.: aerosol nebulization via intra nasal route and oral route) is now being advocated because the stimulation of local immunity. Spray and nebulization of the sub-unit based vaccines and attenuated virus are under clinical scrutiny for assessing induction of virus-specific local immunity able to prevent spreading of the viral infection through person-to-person contacts. This immunization route is also practicable when using viral antigens in VS configuration, given its physical-chemical stability of and its opportune size-dimensions.

### Example 8: Protective Immunity Induced by the Species-Specific VS

The data provided in FIGS. 18A and B demonstrates the active protection by VS Endotoxoid from S. typhimurium LPS against lethal infection in CD1 mice by a homologous strain. Mice are a significant animal model for evaluating the Endotoxoid concept. This protection against an i.p. challenge of Salmonella enterica (serotype typhimurium) was demonstrated in CD1 mice immunized with three doses administered s.c., three weeks apart, of either Endotoxoid Tm or LPS Tm. Control immunization with heterologous LPS (E. coli 055:B5) and saline were performed in parallel. Groups of 20 mice each received a challenge of either LD₁₀₀ (4×10⁵ cells/mouse) (A) or LD₅₀ (9×10³ cells/mouse) (B), three weeks after the third dose of each antigen.

### Example 9: Biological Functionality of the VS Endotoxoid L7 (Group B)—Induced Antibodies in Comparison to a Whole Cell N. meningitidis Group B Vaccine

Four-week-old female CD1 mice were immunized by the s.c. route with 5 μg of Endotoxoid L7 or its protein-conjugate at day 0, 21, 35. Men B strain M982 (L3,L7) as human reference isolate, was previously grown in conditions allowing good expression of LPS antigens and then heat-killed. Negative control (buffer+SAEP) was run in parallel. Blood samples were taken at the retro-orbital sinus and sera analysed by ELISA to detect specific IgG antibodies and biological functionality by complement-mediated serum bactericidal activity (SBA) and complement-mediated opsono-phagocytosis (OP). The results are shown in Table 2.

TABLE 2

| | | Anti-L7 IgG | SBA | OP | Survival rate |
|---|---|---|---|---|---|
| Immunization | | (% responders) | (% responders) | (% responders) | (%) |
| Buffer + SAEP | log⁻¹ | <1.2 | <1.2 | <1.2 | 0 |
| | | (0) | (0) | (0) | |
| Endotoxoid L7 5.0 μg | log⁻¹ | 4.1 | 2.7 | 3.0 | 100 |
| | | (100) | (80) | (90) | |
| CRM197-Endotox L7 5.0 μg | log⁻¹ | 4.7 | 3.1 | 3.2 | 100 |
| | | (100) | (100) | (100) | |
| Whole cell vax 10¹⁰ cell/mouse | log⁻¹ | 4.5 | 3.5 | 3.0 | 100 |
| L3, 7 | | (100) | (100) | (100) | |

Based on the above assessment of murine protection by N. meningitidis LPS L7, immunization in the form of VS Endotoxoid L7 as well as on the established structural antigenic cross-reactivity of such LPS L7 with N. gonorrhoeae LPS which is reported to enhance gonococcal clearance in a murine model of infection, (Matthias K A et al., J.Infect.Dis., 225: 650-660, 2022) one skilled in the art would conclude that VS Endotoxoid L7 can be used to induce protective levels of immunity against N. gonorrhoeae infection opening the door to an effective vaccine against this expanding pathogen.

A similar expectation based on antigenic cross-reactivity is supported by the data in FIG. 19 which illustrates the cross-reactive protection of VS Endotoxoid L7 vaccine (N. meningitidis Group B) offered against a lethal challenge by N. meningitidis Group A (bearing LPS L8 cross-reactive with LPS L7). FIG. 19 illustrates the active cross-protection of VS Endotoxoid L8 (Group A, strain A1 L8) against N. meningitidis Group B (Strain RH 873 L3/L7/L8) at 100LD₅₀/dose.

### Example 10: Biological Functionality of the VS Endotoxoid L8 (Group A)-Induced IgG Antibodies and Cross-Protection Against Challenge by N. meningitidis Group B

Endotoxoid L8-induced protection to bacteraemia due to lethal challenges of pathogenic N. meningitidis Group B bacteria was demonstrated as follows: Four-week-old female CD mice were immunized by the s.c. route with Endotoxoid L8 (A1) at day 0, 21, 35 and then challenged one week after the last injection with 100 LD₅₀ of bacteria [Men B strain 873 (L4,L7,L8) human reference clinical isolate] previously grown in conditions allowing good expression of LPS antigens.

Negative (buffer+SAEP) and positive controls (heat-killed whole cell bacteria homologous to the strain of challenge) were run in parallel. Blood samples were taken at the retro-orbital sinus the day before the challenge and sera analysed by ELISA to detect specific IgG antibodies and biological functionality by complement-mediated serum bactericidal activity (SBA) and complement-mediated opsono-phagocytosis (OP).

The results of these tests are shown in the following Table 3:

TABLE 3

| | | Anti-L8 IgG | SBA | OP |
|---|---|---|---|---|
| Immunization | | (% responders) | (% responders) | (% responders) |
| Buffer + SAEP + FA | log⁻¹ | <1.2 | <1.2 | <1.2 |
| | | (0) | (0) | (0) |
| Endotoxoid L8 2.5 μg | log⁻¹ | 3.0 | 1.5 | 2.3 |
| | | (80) | (30) | (80) |
| Endotoxoid L8 5.0 μg | log⁻¹ | 3.9 | 2.0 | 2.8 |
| | | (90) | (70) | (90) |
| Endotoxoid L8 5.0 μg + FA | log⁻¹ | 4.2 | 2.1 | 3.2 |
| | | (100) | (80) | (100) |
| Whole cell vax 10¹⁰ cell/mouse | log⁻¹ | 4.0 | 3.5 | 2.7 |
| L4, 7, 8 | | (100) | (100) | (80) |

Protective immunity of VS P. aeruginosa (ST 111) was demonstrated in groups of 10 female CD1 mice/each against a systemic bacterial challenge of the pathogen. Both formulations of LPS P. aeruginosa (either VS or Endotoxoid) were injected by i.p. route at the dose of 1 μg LPS.

Immunization doses at week 0, 3, 5 and bacterial challenge (LD₅₀ and LD₁₀₀) at week 6 with an observation period for survival of 2 weeks. Control mice did not receive the vaccine.

The results are shown in the following Table 4:

TABLE 4

| | Challenge | Survived/ |
|---|---|---|
| Immunization by antigen | dose/mouse | Challenged mice |
| VS P. aeruginosa | LD₅₀ (1.5 × 10⁶) | 10/10 |
| | LD₁₀₀ (5 × LD₅₀) | 10/10 |
| Endotoxoid P. aeruginosa | LD50 | 10/10 |
| | LD100 | 9/10 |
| Control (PBS) | LD50 | 4/10 |
| | LD100 | 0/10 |

FIG. 20A provides data that demonstrates the protective effect of VS K. pneumoniae type 1 in CD1 mice against bacterial challenge by homologous strain of K. pneumoniae. Different doses of bacteria were administered to mice, namely, 10⁶, 10⁷ and 10⁸ CFU or a Colony Forming Unit by intraperitoneal injection (i.p.). (mice n=10; *P<0.05 and **P<0.01, compared with the PBS-injected group). FIG. 20B is a survival graph of 10⁸ CFU of K. pneumoniae challenged mice after 3-doses immunization by 0.5, 1 and 2 μg of K. pneumoniae VS at a 1-week interval (mice n=10; ***P<0.001 as compared to the sham mice group).

### Example 11: Immunogenic Properties of the VS Components

As disclosed in this application, the three basic components of the VS are a nanostructured vector system comprising a complex Lipid A-SAEP or a Lipid A derivative-SAEP derivative (Endotoxoid A) that incorporates a helper T-cell dependent carrier protein such as the CRM 197 diphtheria protein or toxoid protein present in the DPT vaccine.

It is rational to expect that each of the three components of the VS express their own level of immunogenicity. In the case of Lipid A and its derivatives, the carbohydrate chain present in the Lipid A derivative (e.g.: The S-LPS of E. coli or the R/S LPS of N. meningitidis or the R-LPS like Re 595) do express a different degree of immunogenicity which actually addresses their relative specificity. Also, the increased expression of immunogenicity for a given Lipid A derivative presented to the immune system of a murine model in VS formulation is shown in FIGS. 21 and 22, based on two different species-specific VS (LPS N. meningitidis L7 and LPS S. minnesota Re 595).

FIG. 21 provides relative immunogenicity data for the VS components as induced by the VS of N. meningitidis L7 or the homologous Endotoxoid (LPS L7-SAEP complex) in comparison to native LPS as reference. The powerful immunogenic activity of the VS L7 formulation with respect to that expressed by the native LPS L7 is well evidenced by the correspondent IgG isotype antibody titers which are ca. 50× (1.7 log) higher. Endotoxoid L7 shows an intermediate immunogenicity with IgG ca. 20× (or 1.3 log) lower than the titers induced by VS.

FIG. 22 provides relative immunogenicity data for the VS components as induced by the VS formulation relative to LPS Re 595 from S. minnesota. A similar Helper T-cell dependent pattern to the one shown in FIG. 21 is observed when LPS Re 595 (as Lipid A derivative) replaces LPS L7 in the VS composition. In contrast, as expected, SAEP shows a very low immunogenic activity (less than 1% of the one expressed by the carrier protein) consistent with the lack of intrinsic immunogenicity of low-MW peptides.

### Example 12: Glycoconjugate-Based Vaccines as VS-Based Vaccines to Achieve Broad-Spectrum Vaccines Against Gram-Positive and/or Gram Negative Bacterial Pathogens

In the paediatric field of preventive medicine, the number of injections/host are ca. 12 in a child 0-5 ys. only for prevention of IPD (Invasive Pneumococcal Diseases) and IMD (Invasive Meningococcal Diseases). By associating different vaccines, when possible, it would simplify the vaccination schedule and save a number of injections to the paediatric community. Accordingly, the species-specific VS N. meningitidis L7, targeting the pathogen N. meningitidis Group B, has been formulated in association with the market-available Quadrivalent Meningococcal Conjugate Vaccine Menveo (GSK)—a polyvalent glycoconjugate vaccine—as an example of a Pentavalent vaccine broadly active against the prevalent meningococcal pathogens belonging to the Group A,B,C,W135,Y According to the actual investigated formulation, all these antigens are based on the Helper-T cell dependent protein CRM197. Immunization of CD1 mice did support the concept of a broad vaccine formulation with no interference among the formulated antigens and with the immunological results reported in FIG. 23.

Comparison of IgG Titers Anti-L7 vs. Anti-Group A,C,Y,W: No Interference Detected in the Immune Response to Each Specific Antigen of the Combined (“COMBO”) Formulation

The strategy of efficiently presenting antigens by the VS of the present invention has been also applied to a new formulation of Polyvalent Pneumococcal Conjugate Vaccine (BiosYnth's U.S. Pat. No. 10,300,135 B2 and EU Patent No. EP2950815 B1) to demonstrate the increased immunogenicity of conventional glycoconjugate antigens targeting Gram-positive pathogens (e.g.: S. pneumoniae) when presented to the immune system of a mammalian through a highly efficient nanostructured formulation.

### Example 13: Immunogenic Properties of a Multi-Valent Glycoconjugate Antigen (the S. pneumoniae Conjugate Triad CRM197—Ps Type 3,6B,14) when Presented to the Immune System of a Mammal by the VS

The above, previously described, multi-valent antigen is a highly immunogenic molecular entity which contains in its composition the Helper T-dependent carrier protein CRM197. This Triad antigen is highly immunogenic “per se” at doses well below 100 ng. In this case, presenting the multi-valent glycoconjugate to the immune system of a mammalian using the VS strategy would likely be of modest immunological benefit given that the Helper T-cell dependent carrier protein CRM197 is already present in the glycoconjugate composition. According to this consideration, and as reported in the previous example shown in FIG. 12, the VS composition for this category of antigens can be conveniently modified by taking advantage of the carrier protein CRM197, which is part of the conjugate composition, as Helper T-cell dependent antigen incorporated within the Endotoxoid A micelle composed of the equimolar complex SAEP-Lipid A (or LPS Re 595). In other words, the conjugated carrier protein CRM197 of the Triad replaces the one originally present in the VS composition. In FIGS. 24A, B and C are reported the immunogenic activity of this VS entity in comparison with the immunogenic activity of the Triad glycoconjugate itself FIGS. 24A, B and C shows the immunogenic activity of the VS incorporating the Triad multivalent glycoconjugate antigen CRM197-Ps 3,6B,14 in comparison to the immunogenic activity of the conjugate itself. Dose of each type-specific Ps in the Triad conjugate is 0.15 μg in both cases, with a VS composition containing 0.41 μg of CRM197 (as glycoconjugate) and 0.84 μg of Endotoxoid A according to the MID-interval reported in FIG. 17. The IgG antibody titers reported are specific for the three type-specific capsular Ps of S. pneumoniae as indicated.

### Example 14: Immunogenic Properties of the VSL7 Compared to the Homologous Endotoxoid L7 and on the Role of a Mineral Adjuvant in Both Formulations

As previously reported in FIG. 7, the Vector System composition when compared to its homologous Endotoxoid does express a higher immunogenicity due to the Helper T-cell activity of the incorporated carrier protein CRM197. While comparing these immunogenic properties in parallel, the role of the mineral adjuvant Al(OH)₃ has been investigated in order to assess its immunogenic potential. Mice have been therefore immunized using the same schedule of immunization as above reported and then assaying the antigen-specific IgG with the results reported below in FIGS. 25A and B.

FIG. 25A shows a comparison of the immunogenic properties of N. meningitidis VS L7 with the homologous Endotoxoid L7 (A) at the comparable dose of 5 μg and FIG. 25B shows the effect played by the mineral adjuvant Al₂(OH)₃ (0.140 mg/dose) on the N. meningitidis Endotoxoid L7 (B).

The immuno-enhanced effect played by the mineral adjuvant on the Endotoxoid L7, as generally known for true Helper T-cell dependent antigens, is well evidenced.

Also evident are the higher immunogenic properties of the VS formulation when compared to the homologous Endotoxoid formulation, an observation explainable with the presence of the Helper T-cell dependent carrier protein CRM197 in the VS composition.

### Example 15: Preclinical Results of the Anti-RBDt Antibody Response Induced by Different VS Formulations

Mice and non-human primates are known to be helpful animal models in establishing the specificity and the neutralizing activity of antibodies raised against CoV-2 by both antigens, the RBD and the whole S-protein (all encompassed within the subunit S1). It is predictable that the same animal models will be helpful in assessing the immunological activity of the disclosed hybrid antigen, or that of each single antigen, and the “ad hoc” developed carrier protein (CRM197) incorporated into the VS. According to the above, a murine model has been used for assessing the immunological concept disclosed in this Application and demonstrate its usefulness in applying it to a conformationally-dependent, structurally fragile, antigen like the RBD peptide structure originating from the original whole sequence of the Spike glycoprotein from SARS-CoV2 virus, the agent of the COVID-19 pathology. Here below, in Table 5, is reported the set of immunological experiments performed in a murine animal model using female CD1 outbred mice.

Table 5 shows the effect of serum IgG pre-titers to the carrier protein CRM197 on the booster activity detected towards the carried RBDt conformational antigen. The hybrid antigen has been administered by the i.p. route at the dose of 1 μg RBDt for any antigen formulation in groups of CD1 female mice (10 animals/group) in parallel to its formulation with the VS. Pre-titers to the carrier protein CRM197 were induced by preliminarily treating the groups of mice with CRM197 alone in order to induce a background serological “memory” as evidenced by the background of IgG to the protein (therefore mimicking a host showing a background of IgG antibodies to at least one of the antigens present in the paediatric DPT vaccine). 2 weeks later, the groups of mice that received pre-treatment by CRM197, were injected with the hybrid antigen CRM197-RBDt alone or in formulation with the VS, in parallel to groups of mice receiving the control antigens during the immunization schedule at week 0, 3, 5 with bleeding before each injection was given and with the final bleeding at week 6. Titers of the sera pool for each group of mice are indicated as log₁₀ value of the end-point dilution in an ELISA assay. The test results are shown in Table 5:

TABLE 5

| | IgG to Antigen RBDt | | | | |
|---|---|---|---|---|---|
| Immunization by Antigen | week −2 | week 0 | week 3 | week 5 | week 6 |
| CRM197 + VS (CRM197-RBDt) | — | — | 5.1 | 5.5 | 5.8 |
| CRM197 + VS (RBDt) | — | — | 4.9 | 5.7 | 5.9 |
| CRM197 + CRM197-RBDt | — | — | 4.2 | 5.1 | 5.2 |
| CRM197-RBDt | — | — | 2.5 | 3.7 | 4.3 |
| RBDt | — | — | 2.3 | 2.5 | 2.6 |
| | IgG to Antigen CRM197 | | | | |
| Immunization by Antigen | week −2 | week 0 | week 3 | week 5 | week 6 |
| CRM197 + VS (CRM197-RBDt) | — | 4.8 | 7.2 | 7.4 | 7.6 |
| CRM197 + VS (RBDt) | — | 4.9 | 6.9 | 7.5 | 7.7 |
| CRM197 + CRM197-RBDt | — | 5.0 | 6.2 | 6.4 | 6.7 |
| CRM197-RBDt | — | — | 4.7 | 5.7 | 6.0 |

The above presented animal data show that a structurally fragile antigen like the isolated RBD sequence present within the whole sequence of the Spike protein of the SARS CoV-2 virus that expresses conformational epitopes, are in good agreement with those obtained with a completely different molecular entity like the VS N. meningitidis L7 reported in FIG. 6 of the present application.

These data do assess for a sub-unit viral antigen like the RBDt peptide-sequence, the immunological principle claimed in the present Application, that is an immunological immediate response (booster effect) is obtained by using a carrier molecular entity like CRM197 vectorizing a (structurally fragile) hapten/antigen when the host immune system has been already primed by a vaccine containing such an antigen. Moreover, the possibility to incorporate the carrier protein and the carried antigen (in conjugated or unconjugated form) into the disclosed Vector System, further increases the immunogenicity of the carried hapten/antigen for a prompt immune response at just the first dose of it.

It has been already demonstrated in the current literature (Routhu N. K. et al. Nature Communications 12.3587, 2021) that antibodies specifically induced to the RBDt antigen are protective against the viral infection induced by the SARS CoV-2 virus bearing the homologous Spike glycoprotein. Also, it is well documented (Yin W. et al. Science 10.1126/science.abn 8863, 2022) that antibodies to RBDt are fine inhibitors of the RBD-ACE2 complex, the first molecular step that allows the infective process to take place.

### Example 16: Extension of the Above Model of Subunit Vaccine for SARS CoV-2 and Endotoxoid A Vector System to Other Pathogenic Viruses

The principle disclosed for this model of subunit vaccine and the Vector System developed can be translated to other viruses reported to express conformational determinants (epitopes), such as:
A vaccine model for dengue 2 virus (flavivirus) that considers a quaternary epitope expressed by the envelop Glycoprotein E (DENV E protein) as target of neutralizing antibodies (see: Kudlacek S T et al. Sci.Adv. (2021) 7: eabg4084)
A vaccine model for the Respiratory Syncytial Virus (RSV) that considers the stabilized pre-fusion F protein or part of its conformational epitopes (see: McLellan J et al Science, 342: 592-598, 2013)
A vaccine model for HIV that considers the outer domain epitope of gp120 that binds to human broadly neutralizing antibodies (bNAbs) (see: Jardine J et al. Science, 340: 711-716, 2013)
A next-generation influenza vaccines targeting multiple antigens HA,NA,NP and M1 for preservation of their native conformations (see: Wei C J et al. Nature Reviews 19: 239-252, 2022)
A next-generation rabies vaccines targeting the virus glycoprotein trimer RABV-G in pre-fusion conformational state expressing a functional quaternary epitope (see: Callaway H M et al. Science Advances 8: eabp 9151, 2022)
Ebolavirus glycoprotein structure and mechanism of entry (see: Lee J E and Saphire E O, Future Virol. 4(6):621-635 (2009)

Such conformational viral antigens of glycoproteic nature can be formulated as single, species-specific, vector systems but also as an association of compositionally defined multiple, species-specific, vector systems that can be assessed as an antigenic mosaic-like formulation when resolved by electron microscopy techniques as previously shown in FIG. 3.

1-29. (canceled)

30. A nanostructured vector system comprising a complex of Lipid A-SAEP (Synthetic Anti Endotoxin Peptide) or a Lipid A derivative-SAEP derivative incorporating at least one helper T-cell dependent carrier antigen, selected from the group consisting of CRM197, Diphtheria Toxoid, Tetanus Toxoid, Pertussis Toxoid or a combination thereof; wherein said Lipid A derivative is an R-LPS or an R/S-LPS or an S-LPS chemotype and wherein said SAEP derivative is a cationic and amphipathic peptide structure in a linear or cyclic conformation with a ratio between cationic and hydrophobic amino acids of ≥0.5, wherein the helper T-cell dependent carrier antigen is not incorporated in the nanostructured vector system via covalent bonds.

31. A nanostructured vector system according to claim 30, wherein the helper T-cell dependent carrier antigen is hydrophobically incorporated in the nanostructured vector system.

32. A nanostructured vector system according to claim 30, wherein the helper T-cell dependent carrier antigen is CRM197.

33. A nanostructured vector system according to claim 30, further comprising one or more bacterial and/or viral antigen, in conjugated or unconjugated form.

34. A nanostructured vector system according to claim 30, wherein said incorporated helper T-cell dependent carrier protein is conjugated with one or more bacterial and/or viral antigens.

35. A nanostructured vector system according to claim 30, wherein the Lipid A derivative-SAEP derivative is Endotoxoid A.

36. A nanostructured vector system according to claim 30, further comprising immunological adjuvants.

37. A nanostructured vector system according to claim 30, for use in medical field as a vaccine to be administered to a mammalian host.

38. A process for the preparation of the nanostructured vector system according to claim 30 comprising the step of combining the Lipid A-SAEP or said a Lipid A derivative-SAEP derivative with an immunogenic helper T-cell dependent carrier protein selected from the group consisting of CRM197, Diphtheria Toxoid, Tetanus Toxoid or Pertussis Toxoid and bacterial and/or viral antigens.

39. A conjugated hybrid antigen comprising a) the bacterial protein CRM197 and b) a viral SARS CoV-2 S-protein expressed by anyone of the SARS CoV-2 S Variants of Concern, said hybrid antigen being in trimeric conformation.

40. A conjugated hybrid antigen according to claim 39, wherein the viral SARS CoV-2 S-protein expressed by anyone of the SARS CoV-2 S Variants of Concern, is the Receptor Binding Domain of said SARS CoV-2 S-protein.

41. The conjugated hybrid antigen according to claim 40, having formula CRM197₃RDB₂ and multiples thereof.

42. A process for the preparation of the nanostructured vector system incorporating the conjugated hybrid antigen according to claim 41, comprising the following steps:
(1) reacting under reducing and then oxidative conditions the helper T-cell dependent carrier protein CRM197 with the viral antigen RBD in order to form covalent, interchain, S—S cross-bridging bonds according to the following stoichiometry of reaction:
3 CRM197(—SH)₂+2 RBDt(—SH)₃
to produce and hybrid antigen having formula CRM197₃RDB₂;
(2) incorporating the hybrid antigen obtained in step 1) into the Lipid A moiety of Endotoxoid A, resulting in the formation of nanoparticles.

43. A vaccine comprising a nanostructured vector system according to claim 30, for the protection of a mammalian host against viral and/or bacterial infections, together with other pharmaceutically acceptable adjuvants.

44. A vaccine comprising a nanostructured vector system according to claim 30 and the hybrid antigen comprising a) the bacterial protein CRM197 and b) a viral SARS CoV-2 S-protein expressed by anyone of the SARS CoV-2 S Variants of Concern, said hybrid antigen being in trimeric conformation or the viral SARS CoV-2 S-protein expressed by anyone of the SARS CoV-2 S Variants of Concern, is the Receptor Binding Domain of said SARS CoV-2 S-protein or the antigen having formula CRM197₃RDB₂ and multiples thereof; for use as a species-specific vaccine for SARS CoV-2 in the prevention of the infection of COVID-19.

45. A vaccine for Dengue 2 virus comprising the nanostructured vector system according to claim 30, where the viral antigen is an envelope Glycoprotein E homodimer, expressing conformational or quaternary epitopes or part of said envelope Glycoprotein E homodimer.

46. A vaccine for Respiratory Syncytial Virus comprising the nanostructured vector system according to claim 30, where the viral antigen is the stabilized RSV prefusion F glycoprotein, or its antigenic conformational site phi containing the RSV F trimer.

47. A vaccine for HIV comprising the nanostructured vector system according to claim 30, wherein the viral antigen is the outer domain epitope of the Glycoprotein gp120, in its native conformation binding to human broadly neutralizing antibodies.

48. A vaccine for Influenza Virus comprising the nanostructured vector system according to claim 30, wherein the viral antigen is at least one of the multiple antigens of the Influenza virus HA, NA, NP and M1 in their respective native conformations.

49. A vaccine for Rabies Virus comprising the nanostructured vector system according to claim 30, wherein the viral antigen is the rabies virus glycoprotein trimer RABV-G in pre-fusion conformational state expressing a functional quaternary epitope.

50. A vaccine for Ebola Virus comprising the nanostructured vector system according to claim 30, wherein the viral antigen is the glycoprotein trimer EBOV in pre-fusion conformational state expressing a functional epitope.

51. A monovalent or polyvalent broad-spectrum vaccine in the human or veterinary field for species-specific bacteria comprising the nanostructured vector system according to claim 30, wherein the bacterial antigens comprise species specific capsular polysaccharides and/or specie specific lipopolysaccharides.

52. A monovalent or polyvalent broad-spectrum vaccine in the human or veterinary field for species-specific bacteria comprising the nanostructured vector system according to claim 30 for use as a species-specific vaccine for Gram-negative and/or Gram-positive bacteria.

53. A monovalent or polyvalent broad-spectrum vaccine in the human or veterinary field for species-specific viral antigens, comprising the nanostructured vector system according to claim 30.

54. A vaccine according to claim 51, wherein the species-specific capsular polysaccharide and/or lipopolysaccharide commonly belongs to the species N. meningitidis and N. gonorrhoeae in a monovalent or multivalent formulation.

55. A vaccine according to claim 51, wherein the species-specific capsular polysaccharides and/or lipopolysaccharides belong to the genus Escherichia, Salmonella, Shigella, Klebsiella, Pseudomonas, Acinetobacter, Staphylococcus or Clostridioides in monovalent or polyvalent formulation for the prevention of Multiple Drug Resistant pathogens.

56. A vaccine comprising at least one nanostructured vector system according to claim 30, formulated in association with one or more polyvalent glycoconjugate vaccine for broad-spectrum immunizations against Gram-positive and/or Gram-negative bacterial pathogens.

57. A vaccine containing more than one nanostructured vector system according to claim 30, by associating multiple vector systems each composed of a species-specific viral antigen, resulting in a broad-spectrum mosaic-like antigenic formulation.

58. A vaccine according to claim 43, in a formulation for parenteral or topical administration in a mammalian host in need thereof.
